(19) United States
(12) Patent Application Publication
(10) Pub. No.: US 2003/0165575 A1
(43) Pub. Date: Sep. 4, 2003

(54) Methods and compositions for treatment and diagnosis of alzheimer disease and other disorders

(75) Inventors: Khalid Iqbal, Staten Island, NY (US); Inge Grundke-Iqbal, Staten Island, NY (US)

Correspondence Address: PENNIE AND EDMONDS, 1155 AVENUE OF THE AMERICAS, NEW YORK, NY, 100362711

(73) Assignee: Research Foundation for Mental Hygeine, Inc., Albany, NY

(21) Appl. No.: 10/218,182

(22) Filed: Aug. 12, 2002

Related U.S. Application Data
(63) Continuation of application No. 09/516,097, filed on Mar. 1, 2000, now abandoned.
Application No. 09/516,097 is a continuation of application No. 08/743,342, filed on Nov. 1, 1996, now abandoned.
Application No. 08/743,342 is a continuation of application No. 08/182,817, filed on Jan. 13, 1994, now abandoned.

(51) Int. Cl. A61K 33/32; A61K 33/06; A61K 38/00

(52) U.S. Cl. 424/639; 424/682; 514/2

(57) ABSTRACT

The present invention is directed to methods for treating Alzheimer disease and other disorders associated with the presence of neurofibrillary tangles (NFTs) by increasing the activity of a phosphatase towards abnormal hyperphosphorylated tau (“AD P-tau”) present in the NFTs of paired helical filaments in the neurons of patients having Alzheimer disease or other NFT-associated disorder. Pharmaceutical compositions and diagnostic methods are also provided. The inventions provide methods of treatment by administering to a subject a therapeutically effective amount of a composition comprising a molecule which increases protein phosphatase activity toward AD P-tau, a phosphatase which dephosphorylates AD P-tau, or a nucleic acid encoding such a phosphatase.

This invention was made in part with government support under grants NS18105, AG05892, AG08076, and AG04220 awarded by the National Institutes of Health. The government has certain rights in the invention.

### 1. INTRODUCTION

The present invention is directed to methods for treatment and diagnosis of Alzheimer disease (AD) and other disorders, and therapeutic and diagnostic compositions. In particular, the invention relates to methods of treatment by administration of molecules which increase the activity of protein phosphatases towards abnormal hyperphosphorylated tau, the major protein subunit of paired helical filaments in neurofibrillary tangles.

### 2. BACKGROUND OF THE INVENTION

### 2.1. Alzheimer Disease

Alzheimer disease, which is the single major cause of dementia in adults in industrialized societies, is a degenerative brain disorder characterized clinically by a progressive loss of memory, confusion, dementia and ultimately death. Histopathologically, Alzheimer disease is characterized by the presence in the neocortex, especially the hippocampus of two brain lesions, the neurofibrillary tangles (NFTs) of paired helical filaments (PHF) in the neurons and the neuritic (senile) plaques of β-amyloid in the extracellular space. In addition to the neurofibrillary tangles in the neuronal perikarya, the PHF also accumulate in the dystrophic neurites surrounding the extracellular deposits of β-amyloid in the neuritic plaques, and in the dystrophic neurites of the neuropil as neuropil threads (Braak et al., 1986, Neurosci. Lett. 65:351-355). Many of the neurons with neurofibrillary changes may be only partially functional, and in some areas of the brain such as neocortex, many of them may eventually die, leaving behind tangled masses of abnormal fibrils, the “ghost tangles”. The β-amyloid also accumulates in the wall and the lumen of the brain vessels. Deposits of β-peptide, polymers of which form amyloid, are also seen as diffuse plaques throughout the affected areas of the brain.

Neither the neurofibrillary tangles nor the plaques are unique to Alzheimer disease. Neurofibrillary tangles of PHF are also found in great abundance in Guam-Parkinsonism dementia complex, dementia pugilistica, postencephalitic parkinsonism, and adults with Down syndrome and in small number in a few cases of subacute sclerosing panencephalitis, Hallervorden-Spatz disease, and neurovisceral lipid storage disease (for review, see Wisniewski et al. 1979, Ann. Neurol. 5:288-294; Iqbal and Wisniewski, 1983, in Alzheimer's Disease, B. Reisberg, ed., The Standard Reference, The Free Press, NY, pp. 48-56). The neuritic (senile) plaques are also seen in Down syndrome and aged humans and in some species of animals. Unlike the tangles, which are present in only very small numbers in non-demented elderly and absent in animals, the plaques are seen frequently in both aged human and animal brains. The numbers of plaques in non-demented aged humans are sometimes similar to those seen in Alzheimer disease cases (Katzman et al., 1988, Ann. Neurol. 23:138-144). Recent studies have shown that most of the plaques found in non-demented elderly, unlike in Alzheimer disease, are free of PHF in the dystrophic neurites (Dickson et al., 1988, Am. J. Pathol. 132:86-101; Barcikowska et al., 1989, Acta. Neuropathol. (Berl.) 78:225-231).

At present, the etiology and the pathogenesis of Alzheimer disease are not established. Alzheimer disease probably has polyetiology, which includes genetic, environmental, and metabolic factors. The major form of Alzheimer disease is sporadic and has a late onset, whereas a small percentage of cases are familial and have an early onset. Some of the familial cases of Alzheimer disease are strongly associated to one or more mutations at different sites on the β-amyloid precursor protein, the gene of which lies on chromosome 21. Whether these mutations are the cause of Alzheimer disease in the affected patients, however, has not been as yet proven experimentally.

### 2.2. Abnormal Phosphorylation of Tau and Disruption of Microtubules

In Alzheimer disease brain there are two general populations of PHF, the PHF I and the PHF II (Iqbal et al., 1984, Acta Neuropathol. (Berl.) 62:167-177). PHF I are readily soluble in sodium dodecyl sulfate, whereas PHF II are solubilized by repeated heat extractions in sodium dodecyl sulfate and β-mercaptoethanol or by ultrasonication followed by extraction in the detergent (Iqbal et al., 1984, Acta. Neuropathol. (Berl.) 62:167-177). PHF I and PHF II probably represent early and late maturation stages, respectively, of the neurofibrillary tangles. The major protein subunit of PHF is the microtubule associated protein tau (Grundke-Iqbal et al., 1986, J. Biol. Chem. 261:6084-6089; Grundke-Iqbal et al., 1986, Proc. Natl. Acad. Sci. USA 83:4913-4917; Grundke-Iqbal et al., 1988, Mol. Brain Res. 4:43-52w Iqbal et al., 1989, Proc. Natl. Acad. Sci. USA 86:5646-5650; Lee et al., 1991, Science 251:675-678). Some of the tau in PHF II and not in PHF I is ubiquitinated (Grundke-Iqbal et al., 1988, Mol. Brain Res. 4:43-52; Morishima-Kawashima-et al., 1993, Neuron 10: 1151-1160; Köpke et al., 1993, J. Biol. Chem. 268:24374-24384).

Tau is a family of several closely related neuronal polypeptides which are generated from a single gene by alternative splicing (Goedert and Jakes, 1990, EMBO J. 9:4225-4230). In adult human brain there are six isoforms of tau which differ from one another in containing three or four tubulin binding repeat domains and the presence or absence of two amino terminal inserts of 29 amino acids, each (Goedert and Jakes, 1990, EMBO J. 9:4225-4230). Tau in PHF is abnormally phosphorylated (Grundke-Iqbal et al., 1986, Proc Natl. Acad. Sci. USA 83:4913-4917; Iqbal et al, 1989, Proc Natl. Acad. Sci. USA 86:5646-5650) The abnormal phosphorylation of tau apparently precedes its polymerization into PHF/neurofibrillary tangles because (a) there is a pool of non-PHF and non-ubiquitinated soluble abnormally phosphorylated tau that can be isolated from Alzheimer disease brain and (b) some of the non-tangle bearing neurons in Alzheimer disease brain and normal aged but not young adult cases are stained immunocytochemically for the abnormal tau (Köpke et al., 1993, J. Biol. Chem. 268:24374-24384; Bancher et al., 1989, Brain Res. 477:90-99; Bancher et al., 1991, Brain Res. 539:11-18).

The abnormally phosphorylated tau from Alzheimer disease brain contains 6-12 moles phosphate per mole of the protein, which is two- to six-fold the level in normal tau; normal tau contains 2-3 moles phosphate per mole of the protein (Iqbal and Grundke-Iqbal, 1991, in Alzheimer's Disease: Basic Mechanisms, Diagnosis and Therapeutic Strategies, Iqbal et al., eds., John Wiley & Sons Ltd., pp. 173-180; Köpke et al., 1993, J. Biol. Chem. 268:24374-24384; Ksiezak-Reding et al., 1992, Brain Res. 597:209-219). To date, nine abnormal phosphorylation sites on PHF tau have been recognized (Table 1).

TABLE 1
Phosphorylation sites of abnormally phosphorylated AD tau

| P-amino | Phos. | | |
|---|---|---|---|
| acid^{a} | Site^{b} | Antibody Used^{c} | Reference |
| Ser 46 | KESP | 102c | Iqbal et al., 1989, Proc. Natl. |
| | | | Acad. Sci. USA 86:5646-5650 |
| Thr 123 | HVTQ | TP30 | Brion et al., 1991, Biochem. |
| | | | J. 273:127-133 |
| Ser 199 | TSSP | Tau-1, AT8 | Grundke-Iqbal et al., 1986, |
| | | | Proc. Natl. Acad. Sci. USA |
| | | | 83:4913-4917; Biernat et al., |
| | | | 1992, EMBO J. 11:1593-1597 |
| Ser 202 | PGSP | Tau-1, AT8 | Grundke-Iqbal et al., 1986, |
| | | | Proc. Natl. Acad. Sci USA |
| | | | 83:4913-4917; Biernat et al., |
| | | | 1992, EMBO J. 11:1593-1597; |
| Thr 231 | VRTP | — | Hasegawa et al., 1992, J. Biol. |
| | | | Chem. 267:17047-17054 |
| Ser 235 | PKSP | SMI33 | Lichtenberg-Kraag et al., |
| | | | 1992, Proc. Natl. Acad. Sci |
| | | | USA 89:5384-5388; Hasegawa |
| | | | et al., 1992, J. Biol. Chem. |
| | | | 267:17047-17054 |
| Ser 262 | IGST | — | Hasegawa et al., 1992, J. Biol. |
| | | | Chem. 267:17047-17054 |
| Ser 396 | YKSP | PHF-1, T3P | Greenberg et al., 1992, J. |
| | | | Biol. Chem. 267:564-569; Lee |
| | | | et al., 1991, Science |
| | | | 251:675-678 |
| Ser 404 | DTSP | ptau 2 | Kanemaru et al., 1992, J. |
| | | | Neurochem. 58:1667-1675 |

^{a}The phosphoamino acid is shown in bold print and underlined.
^{b}The phosphorylation site is numbered according to the largest isoform of human tau, tau₄₄₁.
^{c}The antibody used to map the phosphorylation site.

To date, only phosphorylation of serines and threonines has been shown in normal tau and Alzheimer disease abnormally phosphorylated tau. Phosphoseryl/phosphothreonyl protein phosphatases are classified into four types, termed PP-1, PP-2A, PP-2B and PP-2C (for review, see Cohen, 1989, Annu. Rev. Biochem. 58:453-508). All four protein phosphatases are present in brain tissue (Gong et al., 1993, J. Neurochem. 61:921-927; Ingebritsen et al., 1983, Eur. J. Biochem. 132:297-307; Cohen, 1983, Eur. J. Biochem. 132:297-307). However, it is not known whether the abnormal hyperphosphorylation of tau in AD is a result of an increase of protein kinase activities or an impairment of protein phosphatase activities, or both, or the identities of any such involved kinases or phosphatases. Hence it is essential to identify the protein kinase(s) and phosphatase(s) involved in the regulation of tau phosphorylation. Recently, several protein kinases have been reported to phosphorylate tau in vitro at some of the sites which are abnormally phosphorylated in PHF-tau (e.g. Drewes et al, 1992, EMBO J. 11:2131-2138). However, the identity of the protein phosphatase(s) that can dephosphorylate these abnormal phosphorylation sites are presently not known. Although in vitro several of these phosphorylation sites are accessible to alkaline phosphatase, the overall accessibility to the phosphatase in PHF is less than in normal microtubule tau (Iqbal et al, 1989, Proc Natl. Acad. Sci. USA 86:5646-5650; Iqbal and Grundke-Iqbal, 1990, 1. Neuropathol. Exp. Neurol. 49:270 (Abstract)). The aberrant phosphorylation in Alzheimer disease brains might be selective to a few neuronal proteins and not be a part of a generalized hyperphosphorylation. Levels of both total free phosphate and phosphoprotein phosphate are normal in Alzheimer disease brain (Iqbal et al., 1989, Proc. Natl. Acad. Sci. USA 86:56465650; Iqbal and Grundke-Iqbal, 1990, in Molecular Biology and Genetics of Alzheimer Disease, Miyatake et al., eds., Elsevier, Amsterdam pp. 47-56).

One of the vital functions of the neuron is the transport of materials between the cell body and the nerve endings, and microtubules are required for this axonal transport. Tau stimulates microtubule assembly by polymerizing with tubulin (Weingarten et al., 1975, Proc. Natl. Acad. Sci USA 72:1858-1862) and maintains the microtubule structure (Drubin and Kirschner, 1986, J. Cell Biol. 103:2739-2746). Phosphorylation of tau depresses tau's ability to promote microtubule assembly (Lindwall and Cole, 1984, J. Biol. Chem. 259:5301-5305). In Alzheimer disease brain, the levels of normal tau in the cytosol are decreased by around 40%, whereas the total tissue levels are increased several-fold and this increase is in the form of the abnormally phosphorylated protein (Khatoon et al., 1992, J. Neurochem. 59, 750-753). Binding of guanosine triphosphate (GTP) to the O-subunit of tubulin, which initiates microtubule assembly, is stimulated by tau. Lack of functional tau in Alzheimer disease brain might lead to decreased GTP binding and, consequently, decreased assembly of microtubules (Khatoon et al., 1990, Neurobiol. Aging 11:279 (Abstract)). Microtubules are rarely seen in neurons with neurofibrillary tangles and microtubules are not assembled from brain cytosol of Alzheimer disease cases (Iqbal et al., 1986, Lancet 2:421-426; Iqbal et al., 1987, Lancet 1:102). Both PHF-tau and soluble abnormally phosphorylated tau require dephosphorylation to stimulate in vitro assembly of tubulin into microtubules (Iqbal et al., 1991, J. Neuropathol. Exp. Neurol. 50:316 (Abstract)).

### 2.3. Protein Phosphorylation/Dephosphorylation Systems Involved in Hyperphosphorylation of Tau

The mechanism by which tau in Alzheimer disease brain is abnormally hyperphosphorylated is not as yet established. The state of phosphorylation of substrate proteins depends on the relative activities of protein kinases and phosphoprotein phosphatases.

Seven of the nine phosphorylation sites identified in the abnormally phosphorylated sites to date, are cannonical sites for the proline-directed protein kinases (PDPK). These seven PDPK sites are Ser 46, Ser 199, Ser 202, Ser 231, Ser 235, Ser 396, and Ser 404; the two non-PDPK sites are Thr 123 and Ser 262 (the amino acid numbering is according to the amino acid sequence of the largest isoform of human tau, tau₄₄₁). These findings indicate that most likely more than one protein kinase might be involved in the abnormal phosphorylation of tau in the diseased brain. Phosphorylation of tau at some of the abnormal sites by mitogen-activated protein (MAP) kinases (Roder and Ingram, 1991, J. Neurosci. 11:3325-3343; Drewes et al., 1992, EMBO J. 11:2131-2138), glycogen synthase kinase-3 (Lesdesma et al., 1992, FEBS Lett. 308:218-224; Ishuiguro et al., 1992, J. Biol. Chem 267:10897-10901) and cyclin-dependent cell cycle regulatory kinase, p34^{cdc2} (Vulliet et al., 1992, J. Biol. Chem. 267:22570-22574; Ishiiguro et al., 1992, J. Biol. Chem. 267:10897-10901) have been observed in vitro. However, the time kinetics of the abnormal phosphorylation obtained with these kinases are very slow, requiring up to 24 hours. These findings suggest that either an interaction of the substrate with other protein(s) or a combination of kinases, i.e. site-site interactions might be required for the abnormal phosphorylation of tau.

Studies (Grundke-Iqbal et al., 1986, Proc. Natl. Acad. Sci USA 83:4913-4917; Iqbal et al., 1989, Proc. Natl. Acad. Sci. USA 86:5646-5650; Iqbal and Grundke-Iqbal, 1990, J. Neuropathol. Exp. Neurol. 49:270 (Abstract)) have shown the dephosphorylation of the abnormally phosphorylated sites of tau after treatment with alkaline phosphatase in vitro. The activity of MAP (mitogen activated protein) kinase, which might be involved in the phosphorylation of some of the abnormal sites (Roder and Ingram, 1991, J. Neurosci. 11:3325-3343; Drewes et al., 1992, EMBO J. 11:2131-2138), is inhibited by both PP-2A and phosphotyrosine protein phosphatase (Pelech and Sanghera, 1992, Science 257:1355-1356). Goedert et al. (1992, FEBS Lett. 312:95-99) reported that PP-2A, but not PP-2B could dephosphorylate a MAP-kinase-phosphorylated tau. PP-2A and PP-2B have been shown to dephosphorylate tau phosphorylated by Ca²⁺/calmodulin-dependent protein kinase and cAMP-dependent protein kinase (PKA) (Yamamoto et al, 1988, J. Neurochem. 50:1614-1623; Goto et al., 1985, J. Neurochem. 45:276-283).

Citation of a reference hereinabove shall not be construed as an admission that such reference is prior art to the present invention.

### 3. SUMMARY OF THE INVENTION

The present invention is directed to methods for treating Alzheimer disease and other disorders associated with the, presence of neurofibrillary tangles (NFTs) by increasing the activity of a phosphatase towards abnormal hyperphosphorylated tau (“AD P-tau”). Pharmaceutical compositions and diagnostic methods are also provided

As used herein, AD P-tau shall mean that hyperphosphorylated form of tau as present in the NFT of PHF in the neurons of patients having AD or other NFT-associated disorders (as described in detail in the Examples Sections infra).

The inventions provide methods of treatment by administering to a subject a therapeutically effective amount of a composition comprising (i) a molecule which increases protein phosphatase, (P) activity toward AD P-tau, (ii) a phosphatase which dephosphorylates AD P-tau, or (iii) a nucleic acid encoding such a phosphatase.

### 3.1. Abbreviations

As used herein, the following abbreviations shall have the meanings indicated:

AD=Alzheimer disease

AD P-tau=abnormally phosphorylated tau as found in AD and other disorders associated with NFTs

MAP kinase=mitogen-activated protein kinase

NF=neurofibrillary tangle

PHF=paired helical filaments

PP=protein phosphatase

SDS-PAGE=sodium dodecyl sulfate-polyacrylamide gel electrophoresis

### 4. DESCRIPTION OF THE FIGURES

FIG. 1: A model scheme showing the mechanism of neurofibrillary degeneration in Alzheimer disease. Tau is phosphorylated by several protein kinases including MAP kinase. Because of a decrease in the activities of protein phosphatase 2A (PP-2A) and phosphotyrosine protein phosphatases (PTP) in affected neurons, some of the protein kinases, including the MAP kinase, may remain active for extended periods of time, thereby producing hyperphosphorylated tau. The latter (a) does not bind to tubulin to form microtubules, (b) competes with tubulin in binding to normal tau and inhibits the microtubule assembly, and (c) becomes stabilized and polymerizes into PHF. The affected neurons degenerate both as a result of the breakdown of the microtubule system, and because of the accumulation of PHF as Alzheimer neurofibrillary tangles (ANT) filling the entire cell cytoplasm, leaving behind ghost tangles in the extracellular space.

FIG. 2. Dephosphorylation of AD P-tau by PP-1, PP-2A and PP-2B. Immunoblots of AD P-tau were carried out after incubation either without (lane 1) or with 1.2 units/ml PP-1 (lane 2), PP-2A, (lane 3) or PP-2B (lane 4) at 30° C. for 60 min as described in Section 6.1; lane 5 shows untreated normal human tau for comparison. Reaction mixtures for PP-2A also contained 1.0 mM MnCl₂, whereas for PP-2B, 1.0 μM calmodulin, 1.0 mM CaCl₂ and 1.0 mM NiCl₂ were included. Six phosphorylation-dependent antibodies were used for immunoblotting as shown above each panel. Tau-1, 102c and SMI33 recognize dephosphorylated forms whereas SMI31, SMI34 and PHF-1 recognize phosphorylated forms of tau at specific sites. Molecular weight (kDa) markers are indicated at left of panels.

FIG. 3. Time course of dephosphorylation of AD P-tau by PP-2B. Immunoblots of AD P-tau were carried out after incubation either without (lane 1) or with 1.2 units/ml PP-2B as described in FIG. 2 at 30° C. for different time intervals (lane 2-10); lane 11 is untreated normal human tau. As in FIG. 2, six phosphorylation-dependent antibodies were used to show the site-specific dephosphorylation. Rabbit antiserum, 92e, which recognizes phosphorylation-independent epitopes on tau, was used to show mobility shift. Molecular weight (kDa) markers are indicated at the left.

FIG. 4. Effect of calmodulin and divalent cations on dephosphorylation of AD P-tau by PP-2B. AD P-tau was subjected to immunoblotting with Tau-1 antibody after incubation either without (lane 1) or with 1.2 units/ml PP-2B at 30° C. for different time intervals (lane 2-7); lane 8 contains untreated normal human tau. Dephosphorylation of AD P-tau was carried out in the presence of 1.0 mM MnCl₂ (A); 1.0 mM NiCl₂ (1); 1.0 mM CaCl₂, 1.0 μM calmodulin and 1.0 mM MnCl₂ (C); 1.0 mM CaCl₂, 1.0 AM calmodulin and 10 mM MgCl₂ (D); or 1.0 mM CaCl₂, 1.0 μM calmodulin and 1.0 mM NiCl₂ (E). No apparent dephosphorylation of AD P-tau was observed in the presence of only 1.0 mM CaCl₂ and 1.0 μM calmodulin (not shown).

FIG. 5. Dephosphorylation of AD P-tau by PP-2B at various concentrations of Mn²⁺. AD P-tau was subjected to immunoblotting with Tau-1 antibody after incubation either without (lane 1) or with 1.2 units/ml PP-2B (lanes 2-7), at 30° C. for 60 min as described in Section 6.1 Reaction mixtures also contained 1.0 μM calmodulin, 1.0 mM CaCl₂ and various concentrations (μM) of MnCl₂ as indicated under each lane. Not shown in this Figure is that similar data were obtained when MnCl₂ was substituted with NiCl₂.

FIG. 6. Dephosphorylation of AD P-tau by variable amounts of PP-2A₁, PP-2A₂ and PP-2B. Dephosphorylation of AD P-tau by variable concentration of PP-2A₁ (∘), PP-2A₂ ( [symbol] ) and PP-2B (Δ) was carried out at 30° C. for 30 min, as described in Section 7.1. After reaction, samples were subjected to immunoblotting with Tau-1, and immunoblots were scanned in a densitometer. Dephosphorylation is represented as a percentage of the maximum.

FIG. 7. Immunoblots of AD P-tau after dephosphorylation by variable amounts of PP-2A₁, PP-2A₂ and PP-2B. AD P-tau was subjected to immunoblotting after incubation either without enzyme (lane 1 of each panel) or with 0.5 U/ml(lane 2 of A, D and G), 5.0 U/ml (lane 3 of A, D and G) or 10.0 U/ml (lane 4 of A, D and G) of PP-2A,; 0.5 U/ml (lane 2 of B, E and H), 5.0 U/ml (lane 3 of B, E and H) or 10.0 U/ml (lane 4 of B, E and H) of PP-2A₂; or 0.15 U/ml (lane 2 of C, F and I), 1.5 U/ml (lane 3 of C, F and I) or 3.0 U/ml (lane 4 of C, F and I) of PP-2B. Dephosphorylation reactions were carried out at 30° C. for 30 min, as described in Section 7.1. Antibodies 102c (A. B and C), Tau-1 (D, E and F) and PHF-1 (G, H and D) were used to monitor the dephosphorylation; they recognize Ser-46, Ser-199/Ser-202 and Ser-396, respectively. Molecular weight (kDa) markers are indicated at left of panels.

FIG. 8. Dephosphorylation of AD P-tau at specific sites by PP-2A₁ and PP-2A₂. Immunoblots of AD P-tau were carried out after incubation either without (lane 1) or with 5.0 U/ml PP-2A, (lane 2) Or PP-21A₂ (lane 3) at 30° C. for 60 min, as described in Section 7.1; lane 4 shows untreated normal human tau for comparison. Seven phosphorylation-dependent antibodies and one phosphorylation-independent antibody (92e) were used for immunoblotting, as shown above each panel. Tao-1, 102c and SMI33 recognize dephosphorylated epitopes, whereas AT8, SMI31, SMI34 and PHF-1 recognize phosphorylated epitopes of tau at specific sites, as described in the text. Molecular weight (kDa) markers are indicated at left of panels.

FIG. 9. Treatment of AD P-tau with PP-2A₂ in the presence of okadaic acid and protease inhibitors. Immunoblot of AD P-tau was carried out with mAB Tau-1 (lanes 1-4) or SMI31 (lane 5) after incubation either without (lanes 1 and 5) or with 5.0 U/ml PP-2A₂ (lanes 2-4) at 30° C. for 60 min as described in Section 7.1. Reaction mixtures also included 1.0 M okadaic acid for lane 3 and protease inhibitor cocktail (2.0 μg/ml each of aprotinin, leupeptin and pepstanin, and 2.0 mM benzamidine) for lane 4, respectively. Molecular weight (kDa) markers are indicated at left of the blot. The slowest moving tau band in lanes 2 and 4 is also seen in overloaded normal tau preparations (see Köpke et al., 1993, J. Biol. Chem. 268:24374-24384).

FIG. 10. Time course of dephosphorylation of AD P-tau by PP-2A₁ and PP-2A₂ Immunoblots of AD P-tau were carried out after incubation either without (lane 1) or with 5.0 U/ml PP-2A, (A, C, E and G) or PP-2A₂ (B, D, F and H), as described in Section 7.1 for different time intervals (lanes 2-10); lane 11 is untreated normal human tau. Antibodies 102c (A and B), Tau-1 (C and D), SMI-31 (E and F) and PHF-1 (G and H) were used to monitor the dephosphorylation at Ser-46, Ser-199/Ser-202, Ser-396/Ser-404 and Ser-396, respectively. Molecular weight (kDa) markers are indicated at the left.

FIG. 11. Effect of M²⁺, Mg²⁺ and polylysine on dephosphorylation of AD P-tau by PP-2A₁ and PP-2A₂. AD P-tau was subjected to immunoblotting with Tau-1 antibody after incubation either without (lane 1) or with 5.0 U/ml PP-2A, (A, C, B and G) and 50 U/ml PP-2A₂ (B, D, F and H) at 30° C. for different time intervals (lanes 2-7); lane 8 contains untreated normal human tau. Dephosphorylation of AD P-tau was carried out in the presence of 1.0 mM EDTA (A and B), 2.0 mM MnCl₂ (C and D), 2.0 mM MgCl₂ (E and F) and 10 μM polylysine (G and H).

FIG. 12. PP-1 (upper panel) and PP-2C (lower panel) activities in the presence or absence of various divalent metal ions. Dephosphorylation reactions were carried out using [³²P]phosphorylase kinase as substrate as described in Section 8.1, and in the presence of 1.0 mM EDTA (Δ), 1.0 mM MnCl₂ (▴) or 10 mM MgCl₂ (▪) The open circles (∘) indicate assays in the absence of the protein phosphatases.

FIG. 13. Dephosphorylation of AD P-tau by PP-1, PP-2B and PP-2C Immunoblots of AD P-tau were carried out after incubation either without (lane 1) or with 2.0 units/ml PP-1 (lane 2), PP-2B (lane 3) or PP-2C (lane 4) at 30° C. for 60 min as described in Section 81; lane 5 shows untreated normal human tau for comparison. Reaction mixtures for PP-1 and PP-2C also contained 10 mM MnCl₂ and 10 mM MgCl₂, respectively. For PP-2B, 1.0 μM calmodulin, 1.0 mM CaCl₂ and 1.0 mM MnCl₂ were included. Five phosphorylation-dependent antibodies were used for immunoblotting as shown above each panel to monitor dephosphorylation of the specific sites of AD P-tau. 102c (A), Tau-1 (B) and SMI33 (C) recognize dephosphorylated forms, whereas SMI31 (D) and PHF-i (E) recognize phosphorylated forms of tau at specific sites as described in Section 8.1. Molecular weight (kDa) markers are indicated at left margin of the figure.

FIG. 14. Time course of dephosphorylation of AD P-tau by PP-1. Immunoblots of AD P-tau were carried out after incubation either without (lane 1) or with 10 unit/ml PP-1 as described in FIG. 13 at 30° C. for different time intervals (lane 2-10). Phosphorylation-dependent antibodies Tau-1 (A), SMI31 (B) and PHF-1 (C) were used to monitor the dephosphorylation. Molecular weight (kDa) markers are indicated at the left of each panel.

FIG. 15. Effect of Mn²⁺ and Mg²⁺ on dephosphorylation of AD P-tau by PP-1. AD P-tau was incubated with 10 unit/ml PP-1 in the presence of either 1.0 mM EDTA (∘), 1.0 mM Mn²+(●) or 10 mM Mg²⁺ (▴) at 30° C. for different Lime intervals as described in Materials and Methods. After incubation, AD P-tau was subjected to immunoblotting with monoclonal antibody PHF-1 which stains only phosphorylated forms of tau, followed by densitometric scanning. Dephosphorylation is expressed by percentage of remaining PBF-1 staining.

FIG. 16. Dephosphorylation of PKA-phosphorylated tau by PP:1, PP-2B and PP-2C. PKA-phosphorylated tau (0.1 mg/ml) was incubated either without (∘) or with 0.4 unit/ml of PP-1 (●), PP-2B (▪) or PP-2C (▴) at 30° C. for different time intervals as described in Section 8.1. The reaction mixtures also included 1.0 mM MnCl₂ for PP-1, 10 mM CaCl₂, 1.0 μM calmodulin and 1.0 mM MnCl₂ for PP-2B, and 10 MM MgCl₂ for PP-2C.

FIG. 17. Western Blots of the Cytosolic Acid-soluble Tau and AD P-tau without (A) and with (B) Alkaline Phosphatase Treatment. The amounts of tau loaded onto the gels were 2 μg protein per lane. Blots were immunodeveloped with monoclonal antibody (mAb) Tau-1. Positions of the molecular weight markers in kilodaltons are indicated on the left of panel A. Increased staining on dephosphorylatioin shows abnormally phosphorylated tau. In neither AD nor control cytosolic acid-soluble preparations was any increase in immunostaining seen in the alkaline phosphatase-treated blot (compare panel A with B), suggesting an absence of AD P-tau in these preparations. As expected, the AD P-tau sample was intensely labeled with Tau-1 antibody after alkaline phosphatase treatment of the blot.

FIG. 18. Electron Micrographs Showing the Products of Microtubule Assembly Negatively Stained with Phosphotungstic Acid. Microtubule assembly was carried out from rat brain tubulin by the addition of: a, control acid-soluble tau; b, AD acid-soluble tau; c, AD P-tau; d, AD P-tau after dephosphorylation. Aliquots of each sample were taken at steady state of polymerization. Only an occasional microtubule was seen with tubulin alone (figure not-shown) and with AD P-tau (c), and a large number of microtubules was observed in all of the other situations above (a,b,d). No ultrastructural differences could be seen amongst microtubules assembled with tubulin and normal control tau, AD cytosolic tau or dephosphorylated AD P-tau.

FIG. 19. Effect of praline Phosphatase Treatment on AD Acid-soluble and on AD P-tau on Microtubule Assembly-promoting Activity. The microtubule assembly-promoting activity of AD P-tau (T3) but not of AD acid-soluble tau (A) was increased after the alkaline phosphatase treatment (before, 2; after, 1).

FIG. 20. Effect of AD P-tau on Microtubule Assembly. Polymerization of tubulin was determined as described in Materials and Methods, except that a mixture of normal tau and AD P-tau was used. The assembly reaction was carried out using 0.1 mg/ml of normal tau either mixed with 0.1 mg/ml (4) or 0.2 mg/ml (5) of AD P-tau. For comparison, normal tau was used in different amounts, 01 mg/ml (3), 0.2 mg/ml (2), and 0.3 mg/ml (1). AD P-tau inhibited the microtubule assembly-promoting activity of normal tau (compare curves 2 and 3 with 4; and 1 and 3 with 5).

FIG. 21. Interaction of AD P-tau with Normal Tau and Tubulin. AD P-tau was dotted-on nitrocellulose strips and overlaid with tubulin (▪) or normal tau (●). The nitrocellulose strips were developed with either anti-tubulin antibody DM1A (▪) or with Tau-1 antibody (●). The inset shows the binding of tubulin to normal tau. The amount of tubulin or tau bound is expressed as the relative amount of radioactivity from the radioimmunoassay. Normal tau bound to AD P-tau (●) and tubulin bound to normal tau (inset), but had only background binding to AD P-tau (▪).

FIG. 22. Relationship of the Ratio of Sedimentable Non-hyperphosphorylated Tau/Supernatant Tau (s.nP-tau/sup.tau) to the Levels of AD P-tau. The levels of tau were determined in the 200,000×g supernatant (sup.tau) and 27,000-200,000×g pellet (s.nP-tau and AD P-tau) from brain homogenates of four AD (●) and four control (▪) cases by radioimmuno-slot-blot assay with or without alkaline phosphatase treatment. AD P-tau was calculated from the increase in immunoreactivity after the dephosphorylation (see Section 9.1) The AD P-tau values are expressed as cpm of radioactivity bound per μg of the protein; sn.P-tau/sup.tau ratios were obtained from the means of triplicate assays of these pools of tau determined at two different concentrations. The levels of the non-hyperphosphorylated tau correlate directly with the levels of AD P-tau in the 27,000×g to 200,000×g fraction (Sperman R=0.824, p<0.012), and levels of sup tau in the 200,000×g supernatant correlate inversely with AD P-tau (Sperman R=−0.748, p<0.032). The ratio of sn.P-tau/sup. tau shows a highly significant (Sperman R=0-0.913, p<0.002) direct correlation with the AD P-tau levels.

### 5. DETAILED DESCRIPTION OF THE INVENTION

The present invention is directed to methods for treating Alzheimer disease and other disorders associated with the presence of neurofibrillary tangles (NFTs) by increasing the activity of a phosphatase towards abnormal hyperphosphorylated tau (“AD P-tau”). Pharmaceutical compositions and diagnostic methods are also provided.

As used herein, AD P-tau shall mean that hyperphosphorylated form of tau as present in the NFT of PHF in the neurons of patients having AD or other NFT-associated disorders (as described in detail in tie Examples Sections infra).

As described in the Examples sections infra, it has been discovered that AD P-tau isolated from Alzheimer disease brain is dephosphorylated by the phosphoseryl/phosphothreonyl protein phosphatases PP-2B (calcineurin), PP-2A, and PP-1 (but not PP-2C) and that these enzyme reactivities are markedly increased in the presence of either of the divalent cations Mn²⁺ and Ni²⁺. The order of the level of the phosphatase activity towards AD P-tau is PP-2B>PP-2A>PP-1. Furthermore, PP-2B dephosphorylates the tau abnormally phosphorylated sites Ser 46, Ser 199/Ser 202, Ser 235, Ser 396, and Ser 404, whereas PP-2A dephosphorylates all of these sites except Ser 235, and PP-1 dephosphorylates only Ser 199/Ser 202 and Ser 396.

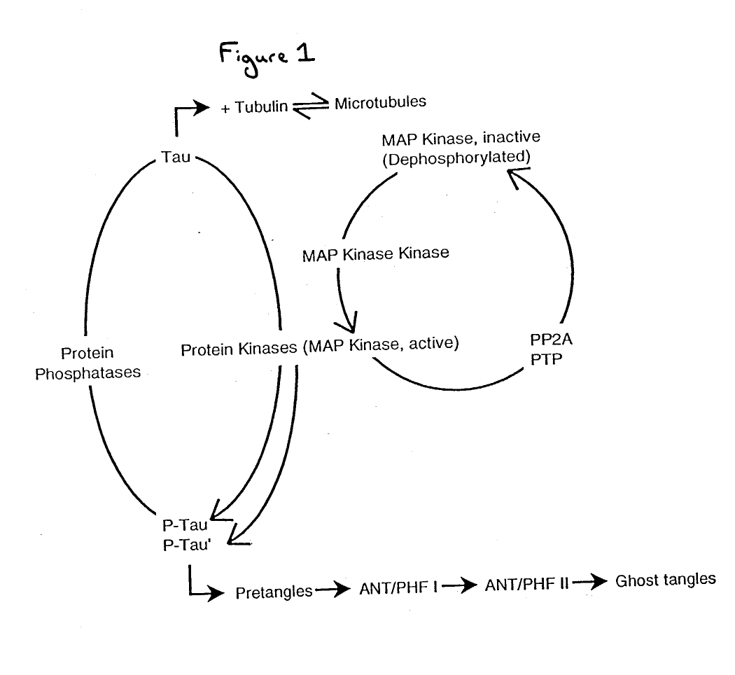

The invention provides various therapeutic methods, which, while not intending to be bound mechanistically, are believed to exert their therapeutic effect by decreasing the level of phosphorylation of AD P-tau, and thus allowing normal microtubule function in the affected neurons of patients. It is believed that (1) that the protein phosphorylation-dephosphorylation system is defective in Alzheimer disease brain, leading to abnormally phosphorylated tau and some other neuronal proteins and (2) that the abnormal phosphorylation of tau contributes to a microtubule assembly defect and consequent impairment of axoplasmic flow and neuronal degeneration (FIG. 1).

The subject which is treated according to the methods of the invention has or is suspected of having a disease or disorder associated with the presence of NFTs of PHF in the neurons. Such a disease or disorder is selected from the group including but not limited to Alzheimer disease, Guam-Parkinsonism dementia complex, dementia pugilistica, postencephalitic parkinsonism, Down's syndrome, subacute sclerosing panencephalitis, Hallervorden-Spatz disease, and neurovisceral lipid storage disease (for a review concerning these disorders, see Wisniewski et al., 1979, Ann. Neurol. 5:288-294; Iqbal and Wisniewski, 1983, Neurofibrillary tangles, in Alzheimer's Disease, Reisberg, B., ed., The Standard Reference, The Free Press, NY, pp. 48-56).

The inventions provide methods of treatment by administering to a subject a therapeutically effective amount of a composition comprising a molecule which increases PP activity toward AD P-tau, a phosphatase which dephosphorylates AD P-tau, or a nucleic acid encoding such a phosphatase. In a preferred aspect, the foregoing therapeutics are substantially purified. In a specific embodiment, administration is repeated over time. The subject is an animal, including but not limited to animals such as cows, pigs, chickens, etc., and is preferably a mammal, and most preferably human. Preferably, the methods of the invention also inhibit the activities of (by dephosphorylating) proline-directed protein kinases such as the MAP kinase, which may participate in the abnormal phosphorylation of AD P-tau.

### 5.1. Administration of Molecules which Increase Protein Phosphatase Activity

In one embodiment, the invention provides methods of treating disorders associated with the presence of NFTs by administering a therapeutically effective amount of a composition comprising a molecule which increases the activity of a protein phosphatase (PP) towards AD P-tau. The PP has the ability to dephosphorylate one or more of the phosphorylation sites of AD P-tau shown in Table 1 hereinabove; the more sites which the PP can dephosphoylate, the more it is preferred. In a preferred aspect, the PP can dephosphorylate at least six of the phosphorylated sites shown in Table 1. In a specific aspect, the PP is selected from the group consisting of PP-1, PP-2A, PP-2B (calcineurin), and related PPs. By “related PPs” is meant PPs which have substantially the same catalytic subunit as one of the foregoing PPs. The terms PP-1, PP-2A, and PP-2B are meant to include the different isotypes for each PP, e.g., PP-2A, and PP-2A₂ for PP-2A. In a specific embodiment, the PP type whose activity is increased according to the invention is generally detectable in neurons of the brain. PP-1, PP-2A and PP-2B are detectable in neurons of the brain, whereas alkaline phosphatase is not. In the methods of the invention, a molecule which increases the activity of PP-2B is most preferred, followed by PP-2A, and then PP-1 in decreasing order of preference. In a preferred aspect, the molecule increases the activity of at least two of the aforesaid PPs, and most preferably all three types. Molecules which can be used therapeutically according to the invention include but are not limited to metals such as Mn²⁺ and Ca²⁺, and polylysine, with Mn²⁺ most preferred. The effect of various metals and of polylysine on AD P-tau dephosphorylation by PP-1, PP-2A, and PP-2B is shown in Table 2.

TABLE 2
Effect of Metal and Polylysine on
AD P-tau Dephosphorylation by PPs

| Effector | PP-1 | PP-2A | PP-2B |
|---|---|---|---|
| Mn²⁺ | ↑ ↑ | ↑ ↑ | ↑ ↑ |
| Mg²⁺ | ↓ | → | ↑ |
| Ni²⁺ | | | ↑ ↑ |
| Ca²⁺ | | | ↑ |
| Al³⁺ | | | ↓ |
| Polylysine | | ↑ | |

↑: Increase in enzyme activity
↓: Decrease in enzyme activity
→: No effect

Other molecules which potentially can be administered for therapeutic effect according to the invention include but are not limited to those listed in Table 3, which are known to increase activity of the indicated PP toward a substrate other than AD P-tau; these potential therapeutics should thus be tested in an appropriate in vitro assay for their effect upon PP activity towards AD P-tau (e.g., in an assay as described in the Examples Sections infra) prior to therapeutic use.

TABLE 3
Protein Phosphatase Activators

| Activator | PP | Reference |
|---|---|---|
| Ceramide | PP-2A (heterotrimer | Dobrowsky et al., 1993, J. |
| | only) | Biol. Chem. |
| | | 268(21): 15523-15530 |
| HSP-70 kDa | PP-1 and/or PP-2A | Mivechi et al., 1993, |
| | | Biochem. Biophys. Res. |
| | | Commun. 192(2):954-963 |
| Insulin | PP-1 | Begum et al., 1993, J. |
| | | Biol. Chem. |
| | | 268(11):7917-7922; Chan |
| | | et al., 1988, Proc. Natl. |
| | | Acad. Sci. USA |
| | | 85(17):6257-6261 |
| Chromostatin | PP-2A | Galindo et al., 1992, Proc. |
| | | Natl. Acad. Sci. USA |
| | | 89(16):7398-7402 |
| cdc2 | PP-1 | Villa-Moruzzi et al., 1992, |
| | | FEBS Lett. |
| | | 304(2-3):211-215 |
| Basic proteins | PP-2A | Ballou and Fischer, 1987, |
| | | The Enzyme 17:311-361 |
| Polyamines | PP-2A | Ballou and Fischer, 1987, |
| | | The Enzyme 17:311-361 |
| Insulin | PP-2A | Speth and Lee, 1984, J. |
| | | Biol. Chem: 259:4027-4030 |
| Calpain | PP-2B | Wang et al., 1989, |
| | | Biochem. Cell Biol. |
| | | 67(10):703-711 |
| Growth factor | PP-1 | Chan et al, 1988, Proc. |
| | | Natl. Acad. Sci. USA |
| | | 85(17):6257-6261 |
| Spermine | PP-2A | Damuni et al., 1987, J. |
| | | Biol. Chem. |
| | | 262(11):5133-5138 |
| Trypsin | PP-2B | Wolff and Sued, 1985, J. |
| | | Biol. Chem. |
| | | 260(7):4195-4202 |

In a specific embodiment, the molecule increases the activity towards AD P-tau of at least one of the foregoing PPs' and does not inhibit any such activity of the foregoing PPs. In another specific embodiment, the molecule is not Mg²⁺. The metal can be in ionic form, salt form, or conjugate. The manganese is preferably in the form of a water-soluable salt such as but not limited to manganese chloride, manganese sulfate, manganese acetate, manganese gluconate, manganese lactate, and manganese citrate. The manganese may also be in the form of other compounds such as manganese hypophosphite, manganese silicate, manganese sulfide, manganese iodide, manganese phosphate, manganese borate, manganese bromide, manganese oleate, manganese nitrate, manganese carbonate, manganese carbonyl, manganese difluoride, manganese trifluoride, manganese oxalate, manganese oxide, manganese dioxide, manganese selenide, manganese sesquioxide, etc. In a specific embodiment, the manganese is not in the form of manganese pyruvate or a manganese chelate of an alkylamino-ester of phosphoric acid (e.g., manganese aminoethyl phosphate). Preferably, such molecules are administered orally, although any form of administration known in the art can be used (see Section 5.5 infra).

Molecules which increase the activity of a PP toward AD P-tau can be identified as having such activity by any appropriate in vitro assay, preferably an assay described in the Examples Sections infra.

Molecules demonstrated to have the desired activity in vitro can then be tested further in vitro if desired, and then in vivo to demonstrate therapeutic efficacy. For example, such molecules can be tested in suitable cell culture systems for their effect on AD P-tau in cultured cells, and in animal systems prior to testing in humans, including but not limited to rats, mice, chicken, cows, monkeys, rabbits, etc. Suitable model systems, where available in the art, can be used.

### 5.2. Administration of Protein Phosphatases and Nucleic Acids Encoding the Same

In another embodiment, the invention provides methods of treating disorders associated with the presence of NFTs by administering a therapeutically effective amount of a phosphatase which dephosphorylates (at least some phosphorylated residues of) AD P-tau. Such a phosphatase preferably is active toward AD P-tau to a greater extent than normal tau. In a specific aspect, such a phosphatase is selected from the group consisting of PP-1, PP-2A, PP-2B, related PPs, and functionally active derivatives and analogs thereof. By “related PPs” is meant PPs which have substantially the same catalytic subunit as one of the foregoing PPs. The terms PP-1, PP-2A, and PP-2B are meant to include the different isotypes for each PP, e.g., PP-2A, and PP-2A₂ for PP-2A. In a specific embodiment, the phosphatase which is administered is a PP whose activity can be detected in neurons of the brain. PP-2B is most preferred for use, followed by PP-2A and then PP-1 in decreasing order of preference. In specific embodiments, one, two, or three of the aforesaid phosphatases can be administered in combination.

Phosphatases can be purified from biological sources by methods known in the art (see e.g., Examples Sections infra), or purchased where commercially available (see Examples Sections infra), or expressed by recombinant methods known in the art from host cells containing a cloned gene encoding a PP. Functionally active derivatives and analogs can be obtained by chemical or enzymatic modification of the phosphatase (e g., acetylation, carboxylation, amidation, phosphorylation, cleavage, etc.) or recombinant manipulation of the gene encoding a PP, all by methods commonly known in the art.

PPs which have the desired activity toward AD P-tau can be identified by an in vitro assay such as described in the Examples Sections infra.

In another embodiment, nucleic acids encoding one or more of the aforesaid PPs can be administered in vivo such that the encoded PP is expressed for therapeutic effect. The cloning and/or nucleotide sequences of PPs are available in the art, e.g., as described in the following publications. For PP-1: Sasaki et al, 1990, Jpn. J. Cancer Res. 81: 1272-1280. For PP-1α: Berndt et al., 1987, FEBS Lett. 223:340-346. For PP-2A: Kitagawa et al., 1988, Biochim. Biophys. Acta 951:123-129; Kitagawa et al., 1988, Biochem. Biophys. Res. Commun. 157:821-827; Sasaki et al., 1990, Biochem. Biophys. Res. Commun. 170:169-175. For PP-2B: Muramatsu and Kincaid, 1993, Biochim. Biophys. Acta 1178:117-120; Guerini and Klee, 1989, Proc. Natl. Acad. Sci. USA 86:9183-9187; Kincaid et al., 1990, J. Biol. Chem. 265:11312-11319; Kincaid et al, 1988, Proc. Natl. Acad. Sci. USA 85:8983-8987.

### 5.3. Methods of Diagnosis

The present invention also provides methods of diagnosing the presence, staging the progression, and monitoring treatment of diseases and disorders associated with the presence of NFTs by detecting or measuring the levels of AD P-tau in a sample from a subject having or suspected of having such a disease or disorder. Preferably, the sample is cerebrospinal fluid (CSF), which can be obtained by a spinal tap as commonly performed in the art. The detection or measurement of AD P-tau levels is preferably carried out by contacting any AD P-tau in the sample with an antibody (or antibodies) which specifically bind to phosphorylated epitopes of AD P-tau (and do not substantially bind to nonphosphorylated epitopes of tau) such that immunospecific binding can occur, and detecting or measuring the amount of immunospecific binding that occurs. An increased level of AD P-tau which is thus observed relative to subjects not having the disease or disorder indicates the presence of the disorder in the subject. Increased levels over time indicate disease progression. It is believed that decreased levels after treatment will indicate treatment efficacy.

The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. In specific embodiments, the immunoassay is carried out by sandwich immunoassay, immunoprecipitation, or dot blot methods, as are well known in the art. Antibodies which can be used, which recognize phosphorylated epitopes of AD P-tau, are known in the art and include but are not limited to those listed in Table 1, Section 2.2 hereinabove, or described in the Examples Sections infra. Antibodies can also be generated by standard methods commonly known in the art, by use of AD P-tau as immunogen.

In one embodiment, the sandwich assay is carried out by binding (as capture antibody) an antibody which recognizes AD P-tau (which need not be specific to a phosphorylated epitope) to a solid substrate (e.g. a plastic dish), incubating with sample (e.g. CSF); and incubating with (as detection antibody) an antibody which specifically recognizes a phosphorylated epitope of AD P-tau. Sub stances which do not immunospecifically bind are removed by one or more washing steps, commonly known in the art.

In another specific embodiment, the dot blot approach, the biological sample (e.g., CSF or proteins obtained therefrom) is applied to a membrane filter, washed, and then contacted with a composition containing the antibody to a phosphorylated epitope of AD P-tau.

In one embodiment, the antibody which binds to the phosphorylated epitope of AD P-tau is labeled (e.g., by an enzyme, radionuclide, fluorescent tag), and the presence of the label is detected or measured. In another embodiment, such antibody is unlabeled, and a labeled specific binding partner to the antibody is added, allowed to bind to the antibody, preferably a washing step is performed, and then the label of the binding partner is detected or measured.

The antibody(ies) can be polyclonal or monoclonal.

### 5.4. Pharmaceutical Compositions

The present invention also provides pharmaceutical compositions. Such compositions comprise a therapeutically effective amount of a therapeutic of the invention (a molecule which increases the activity of a PP toward AD P-tau, a phosphatase which dephosphorylates AD P-tau, or a nucleic acid encoding such a phosphatase), and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, cellulose, etc. Examples of suitable pharmaceutical carriers are described in “Remington's Pharmaceutical Sciences” by E. W. Martin. Such compositions will contain a therapeutically effective amount of the therapeutic, preferably in purified form, together with a suitable amount of carrier so as to provide the form for proper administration to the patient. The formulation should suit the mode of administration.

In a specific embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The therapeutics of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2 ethylamino ethanol, histidine, procaine, etc.

The amount of the therapeutic of the invention which will be effective in the treatment of a particular disorder or condition will depend on the nature of the disorder or condition, and can be determined by standard clinical techniques. In addition, in vitro assays may optionally be employed to help identify optimal dosage ranges. The precise dose to be employed in the formulation will also depend on the route of administration, the physical condition of the subject, and the seriousness of the disease or disorder, and should be decided according to the judgment of the practitioner and each patient's circumstances. However, suitable dosage ranges for intravenous protein administration are generally about 20-500 micrograms of active molecule per kilogram body weight. Suitable dosage ranges for intranasal administration of protein are generally about 0.01 μg/kg body weight to 1 mg/kg body weight. Effective doses may be extrapolated from the dose-response curves derived from in vitro or animal model test systems.

Suppositories generally contain active ingredient in the range of 0.5% to 10% by weight; oral formulations preferably contain 10% to 95% active ingredient.

In a preferred aspect in which manganese is the therapeutic, the dosage of a composition comprising manganese (in salt or conjugate form) which is administered is so as to achieve a level of Mn²⁺ in the brain greater than 10 μM (basal levels of Mn²⁺ in the brain are about 6-10 μM), and preferably so as to achieve a level of Mn²⁺ in the brain in the range of 20-100 μM, and most preferably 40-100 μM. The dosages for achieving 20-100 μM Mn²⁺ concentration in the brain are believed to be in the range of 2.5-12.5 mg manganese compound/kg body weight/day when oral administration is used to achieve 40-100 μM Mn²⁺ concentration in the brain, the dosages are believed to be in the range of 5-25 μg manganese compound/kg body weight/day when intravenous administration is used.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

### 5.5. Therapeutic Administration

Various delivery systems are known and can be used to administer a therapeutic of the invention, e.g., encapsulation in liposomes, microparticles, microcapsules, expression by recombinant cells, receptor-mediated endocytosis (see, e.g., Wu and Wu, 1987, J. Biol. Chem. 262:4429-4432), construction of a therapeutic nucleic acid as part of a retroviral or other vector, etc. The therapeutics of the invention, particularly those with the ability to cross the blood-brain barrier (e.g., manganese, nickel), can be administered systemically, and more preferably parenterally, i.e., via an intraperitoneal, intravenous, perioral, subcutaneous, intramuscular, intraarterial, etc. route, in order to treat disease. Methods of introduction include but are not limited to intradermal, intramuscular, intraeritoneal, intravenous, subcutaneous, intranasal, epidural and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. In a specific aspect, the compounds are directly administered to the cerebrospinal fluid by intraventricular injection. Pulmonary administration can also be employed, e.g., by use of an inhaler or nebulizer, and formulation with an aerosolizing agent (for example, in the administration of manganese ion, or protein therapeutic, etc.).

In a specific embodiment, it may be desirable to administer the therapeutics of the invention locally to the area in need of treatment; this may be achieved by, for example, and not by way of limitation, local infusion during surgery, topical application, by injection, by means of a catheter, by means of a suppository, or by means of an implant, said implant being of a porous, non-porous, or gelatinous material, including membranes, such as sialastic membranes, or fibers.

In another embodiment, the therapeutic compound can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp 353-365 (1989); Lopez-Berestein, ibid., pp. 317-327; see generally ibid.)

In yet another embodiment, the therapeutic compound can be delivered in a controlled release system. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J. Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-138 (1984)).

Other controlled release systems are discussed in the review by Langer (Science 249:1527-1533 (1990)).

In a specific embodiment where the therapeutic is a nucleic acid encoding a protein phosphatase, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et alt, 1991, Proc. Natl. Acad. Sci USA 88:1864-1868), etc. Alternatively, a nucleic acid therapeutic can be introduced intracellularly and incorporated within host cell DNA for expression, by homologous recombination.

### 6. EXAMPLE: ALZHEIMER DISEASE ABNORMALLY PHOSPHORYLATED TAU IS DEPHOSPHORYLATED BY PROTEIN PHOSPHATASE-2B (CALCINEURIN)

As described herein, we have examined the site-specific dephosphorylation of abnormal hyperphosphorylated tau (AD P-tau) from Alzheimer disease brains by different protein phosphatases. Tau dephosphorylation was monitored by its interaction with several phosphorylationdependent antibodies. AD P-tau was dephosphorylated by brain protein phosphatase-2B at the abnormally phosphorylated sites Ser-46, Ser-199, Ser-202, Ser-235, Ser-396 and Ser404, and its relative mobility on SDS-PAGE shifted to that of normal tau. Protein phosphatases-1 and -2A could dephosphorylate only some of the above six phosphorylation sites.

### 6.1. Material and Methods

AD abnormally phosphorylated tau and normal human tau were isolated from autopsied brains as described by Köpke et al. (1993, J. Biol. Chem. 268:24374-24384). Phosphorylase kinase was purified from rabbit skeletal muscle by the method of Cohen (1973, J. Biochem. 34:1-14). Rabbit skeletal muscle PP-1 was purchased from Upstate Bioteehnology Inc., Lake Placid, N.Y. Rat brain PP-2A₁ and PP-2A₂ were kindly provided by Dr. S. Jaspers of University of Massachusetts. PP-2B (holoezyme) was purified from bovine brain according to the method of Sharma et al. (1983, Meth. Enzymol. 102:210-219). Phosphorylase and calmodulin were purchased from Sigma, St. Louis, Mo. Polyclonal antibodies 102c and 92e were raised as previously reported (Grundke-Iqbal et al, 1988, Mol. Brain Res. 4:43-52; Iqbal et al., 1989, Proc. Natl. Acad. Sci. USA 86:5646-5650). Monoclonal antibodies Tau-1 and PHF-1 were kindly provided by Drs L. I. Binder (Binder et al., 1985, J. Cell Biol. 101:1371-1378) and S. Greenberg (Greenberg et al., 1992, J. Biol. Chem. 267:564-569), respectively; SMI33, SMI31 and SMI34 were purchased from Sternberger Monoclonals Inc., Baltimore, Md. Alkaline phosphatase-conjugated goat anti-mouse and anti-rabbit IgG were purchased from Bio-Rad, Hercules, Calif.

Phosphorylase (2.0 mg/ml) was phosphorylated in 40 mM Tris-HCl, pH 8.5, 20 mM β-mercaptoethanol, 0.2 mM CaCl₂, 15 mM MgCl₂, 10 μg/ml phosphorylase kinase and 0.5 mM [γ-³²P]ATP. After incubation at 30° C. for 10 min, [³²P]phosphorylase (0.9 mol ³²P incorporated/95,000 g) was separated from free ATP on Sephadex G-50 column. [³²P]phosphorylase kinase (1.9 mol ³²P incorporated/95,000 was prepared as reported previously. (Gong et al.:, 1993, J. Neurochem. 61:921-927). The activities-of PP-1, PP-2A and PP-2B were measured by-counting the radioactivity released from [³²P]substrate as previously described (Gong et al, 1993, J. Neurochem. 61:921-927). The reaction mixtures contained 50 mM Tris, pH 7.0, 20 mm β-mecptoethanol, 2.0 mM MnCl₂ and 2.0 μM [³²P]phosphorylase for PP-1 and PP-2A; and 50 mM Tris, pH 7.0, 20 mM β-mecaptoethanol, 10 mM CaCl₂, 10 μM calmodulin and 10 μM [³²P]phosphorylase kinase for PP-2B One unit of protein phosphatase activity is defined as that amount which catalyzes the release of 1.0 mmol phosphate per min from [³²P]substrate at 30° C.

Unless otherwise stated, dephosphorylation of AD P-tau was carried out at 30° C. in 50 mM Tris, pH 7.0, 10 mM β-mecaptoethanol, 0.1 mg/ml BSA, 50 μg/ml AD P-tau and PP-1 PP-2A or PP-2B. The reaction was started by addition of enzyme and stopped by addition of 5 volumes of cold acetone. The precipitated protein samples were dissolved in sodium dodecyl sulfate-polyacrylamide gel electrophoresis (SDS-PAGE) sample buffer and heated at 95° C. for 4 min, followed by 10% SDS-PAGE. Immunoblotting was carried out as described previously (Grundke-Iqbal et al., 1986, Proc. Natl. Acad. Sci. USA 83:4913-4917). The primary antibodies for immunoblotting and their epitopes have all been previously characterized. Antibodies 102c (Iqbal et al., 1989, Proc. Natl. Acad. Sci. USA 86:5646-5650), Tau-1 (Biernat et al, 1992, EMBO J. 11:1593-1597; Grundke-Iqbal et al., 1986, Proc. Natl. Acad. Sci. USA 83:4913-4917) and SMI33 (Lichtenberg-Kraag et al., 1992, Proc. Natl. Acad. Sci. USA 89.5384-5388) recognize dephosphorylated form of tau at sites Ser-46, Ser-199/Ser-202 and Ser-235 respectively. While antibodies SMI31, SMI34 (Lichtenberg-Kraag et al., 1992, Proc. Natl. Acad. Sci. USA 89:5384-5388) and PBF-1 (Lang et al., 1992, Biophys. Res. Commun. 187:783-790) recognize tau phosphorylated at Ser-396/Ser404, Ser-235/Ser-396 and Ser-396, respectively. Antiserum 92e is a phosphorylation-independent antibody which recognizes both AD P-tau and normal tau. These antibodies were used at dilution of 1:100 for 102c, SMI31 and SMI34, 1:500 for SMI33 and PHF-1, 1:5,000 for 92e, and 1:500,000 for Tau-1.

### 6.2. Results

Dephosphorylation of AD Abnormally Phosphorylated Tau (AD P-Tau) by Different Protein Phosphatases

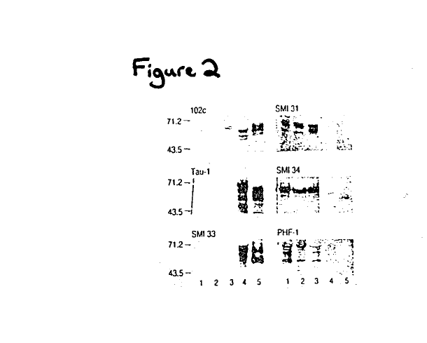

PP-2B (1.2 units/ml) was found to change epitopes of all six antibodies (FIG. 2), suggesting that it can dephosphorylate AD P-tau at Ser-46, Ser-199, Ser-202, Ser-235, Ser-396 and Ser4. Equivalent activities (1.2 units/ml) of PP-1 and PP-2A did not change the epitopes appreciably. After prolonged incubation with PP-1, PP-2A₁ or PP-2A₂ (1.2 units/ml) or after the addition of 1.0 mM Mn²⁺, the epitopes of some of the six antibodies were changed (data not shown). These results indicate that PP-1 and PP-2A can dephosphorylate only some of above six abnormal phosphorylated sites studied. Thus PP-2B seems to be a preferential AD P-tau phosphatase. Hence the dephosphorylation of AD P-tau only by PP-2B was further examined in this study.

Time Course of Dephosphorylation of AD P-Tau by PP-2B

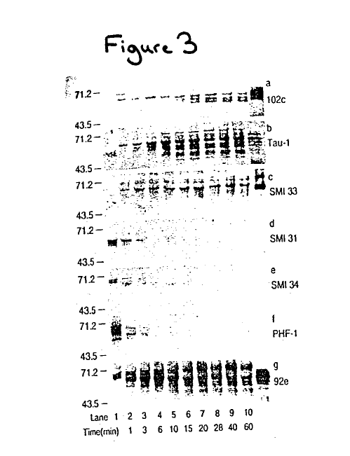

The rates of dephosphorylation of various sites were rapid but nonidentical (FIG. 3). After only 1 min incubation, the staining of tau by 102c, Tau-1 and SMI33 was already apparent, and staining by SMI31, SMI34 and PHF-1 began to disappear Epitopes of SMI33, Tau-1 and 102c were almost completely unblocked in 3 min, 15 min and 20 min, respectively. Blocking of the epitopes of antibodies SMI31, PHF-1 and SMI34 was practically complete in 6 min, 6 min and 15 min, respectively. These results indicate that in hyperphosphorylated AD P-tau the preferential substrate sites for PP-2B are pSer-235>pSer-396/pSer404>pSer-199/pSer-202>pSer-46.

Phosphorylated and dephosphorylated tau are known to have different relative mobilities on SDS-PAGE (Grundkec-Iqbal et al., 1986, Proc Natl. Acad. Sci. USA 83:49134917). After only 1 min incubation with PP-2B, a mobility shift of different tau species is already apparent. Maximal shift of all isoforms, except the slowest one, is achieved in 15 min (FIG. 3, a, b and g).

Effects of Calmodulin and Divalent Cations on Dephosphorylation of AD P-tau by PP-2B

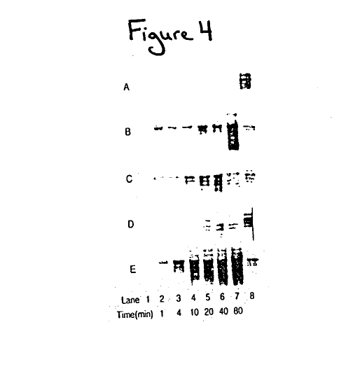

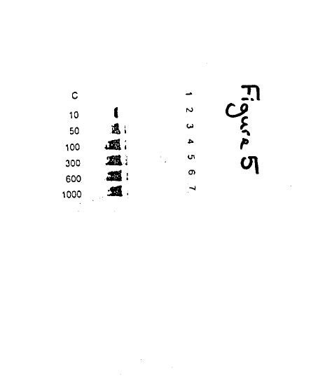

We have investigated the effects of different divalent cations on the dephosphorylation of AD P-tau by PP-2B (FIG. 4). The highest activity of PP-2B towards AD P-tau was observed in the presence of Ca²⁺, Ni²⁺ and calmodulin (FIG. 4, E). When Mn²⁺ and Mg²⁺ were used instead of Ni²⁺, the activity was lower (FIG. 4, compare C and D with E). In the presence of either Mn²⁺ or Ni²⁺ alone, the magnitude of dephosphorylation was lower compared to the inclusion of Ca²⁺ and calmodulin in reaction mixtures (FIG. 4, compare A with C and B with E). In the presence of Ca²⁺ and calmodulin alone, however, no apparent dephosphorylation of tau by PP-2B was detected (data not shown). We further examined the required concentration of divalent metal ions for the dephosphorylation of AD P-tau by PP-2B and found that in the presence of 1.0 mM Ca²⁺ and 1.0 μM calmodulin, 10 FM of either Mn²⁺ or Ni²⁺ activated the dephosphorylation. Maximal dephosphorylation was achieved in the presence of 100 μM of either Mn²⁺ or N²⁺ (FIG. 5).

### 6.3. Discussion

In the present study, we found that PP-2B can dephosphorylate all the six abnormal phosphorylation sites studied as well as change relative electrophoretic mobility of AD P-tau into a normal state. This in vitro dephosphorylation required the presence of 10-100 μM of either Mn²⁺ or Ni²⁺. The physiological level of Mn²⁺ was reported as 5-11 μM in brain (Friberg et al., 1986, Handbook on the toxicology of methods, Vol. 2, Nordberg and Vouk, eds., Elsevier Science Publishers, New York, pp. 264-366), and therefore the dephosphorylation of AD P-tau by PP-2B may have physiological significance. This study indicates that PP-2B might be involved in dephosphorylation of AD P-tau in vivo. The following observations from other laboratories strengthen this suggestion. First, PP 2B has the highest expression in brain, and the amount of PP-2B in brain tissue is about 1% of total proteins, higher than that of other protein phosphatases (Cohen, 1989, Annu. Rev. Biochem. 58:453-508; Kuno et al., 1992, J. Neurochem. 58:1643-1651). Second, immunohistochemical and immunochemical studies have shown that PP-2B is located on microtubules and is plentiful in the cerebral cortex and hippocampus (Kuno et al., 1992, J. Neurochem. 58:1643-1651). Third, it was recently reported that PHF-like tau was generated after incubation of fresh brain slices with 5-20 μM okadaic acid (which inhibits PP-2B as well as PP-2A and PP-1), and that PP-2B can reverse the effect of okadaic acid (Harris et al., 1993, Ann Neurol 33:77-87).

Goedert et al (1992, FEBS Lett. 312:95-99) have reported that MAP kinase-phosphorylated tau could be dephosphorylated by PP-2A₁, one of three subtypes of PP-2A, but not by PP-2B. A cause of this discrepancy between these two studies might be the use of different substrates. AD P-tau (physiological substrate) (which we employed) and MAP kinase-phosphorylated tau which requires 16-24 hours for in vitro phosphorylation (Drewer et al., 1992, EMBO J. 11:2131-2138; Goedert et al., 1992, FEBS Lett. 312:95-99) might behave as different substrates for PP-2B. This is because only some and not all abnormal sites are phosphorylated by MAP kinase (Biernat et al., 1993, Neuron 11:153-163), and non-MAP kinase sites might alter the kinetics of the dephosphorylation.

In a previous study measuring protein phosphatase activities using [³²P]phosphorylase kinase as substrate (Gong et al., 1993, J. Neurochem. 61:921-927), we found that PP-1 and PP-2A activities were significantly decreased in AD brains as compared with controls. We did not, however, find such a decrease in activity of PP-2B. One possible reason for this may be that PP-2B activity was not measured using tau as substrate

### 7. EXAMPLE: DEPHOSPHORYLATION OF ALZHEIMER DISEASE ABNORMALLY PHOSPHORYLATED TAU BY PROTEIN PHOSPHATASE-2A

As described herein, the site-specific dephosphorylation of abnormally phosphorylated Alzheimer tau (AD P-tau) by protein phosphatase-2A was examined by its interaction with several phosphorylation-dependent antibodies to various abnormal phosphorylation sites. Protein phosphatase-2A was able to dephosphorylate AD P-tau at Ser-46, Ser-199, Ser-202, Ser-396, and Ser-404, but not at Ser-235 (the amino acids are numbered according to the largest isoform of human tau, tau₄₄₁). Two major types of protein phosphatase-2A—PP-2A₁ and PP-2A₂—dephosphorylated AD P-tau at approximately the same rate. After AD P-tau was dephosphorylated by protein phosphatase-2A, its relative mobility on SDS-PAGE increased. The dephosphorylation of AD P-tau by PP-2A, and PP-2A₂ was markedly stimulated by Mn²⁺. These results indicate that tau dephosphorylation is catalyzed by both types of protein phosphatase-2A, PP-2A₁ and PP-2A₂.

Several protein kinases have been reported to phosphorylate tau in vitro at some of the same sites at which PHF-tau is abnormally phosphorylated (Drewes et al., 1992, EMBO J. 11:2131-2138; Ishiguro et al., 1992, J. Biol. Chem 267:10897-10901; Ledesma et al., 1992, FEBS Lett. 308:218-224; Mandelkow et al., 1992, FEBS Lett 314:315-321; Vulliet et al., 1992, J. Biol. Chem. 267:22570-22574). However, the in vitro conditions required to obtain the abnormally hyperphosphorylated tau identical to that in Alzheimer disease are still unknown. Hence, we used hyperphosphorylated tau isolated from Alzheimer brain as a substrate to study the potential protein phosphatases that may be involved in the dephosphorylation of tau. Monitoring by immunoblotting with several phosphorylation-dependent antibodies 102c, Tau-1, SMI33, SMI31, SMI34 and PHF-1, we found that protein phosphatase-2B (PP-2E3) can rapidly dephosphorylate Alzheimer tau at sites SerA6, Ser-199, Ser-202, Ser235, Ser-396 and Ser-404 in vitro (see Section 6 above). In this Section, we report that protein phosphatase-2A (PP-2A), in addition to PP-2B, can dephosphorylate AD P-tau at phosphorylation sites Ser-46, Ser-199, Ser-202, Ser-396 and Ser-404 but not at Ser-235

### 7.1. Experimental Procedures

Materials

Phosphorylase kinase was purified from rabbit skeletal muscle by the method of Cohen (1973, Eur. J. Biochem. 34:1-14). PP-2A₁ and PP-2A₂ were purified from rat brain basically as described by Cohen et al. (1988, Methods in Enzymol. 159:390-408) and kindly provided by Dr. S. Jaspers of the University of Massachusetts. PP-2B was purified from bovine brain according to the method of Sharma et al. (Sharma et al., 1983, Methods Enzymol. 102:210-219). Phosphorylase, calmodulin, and poly-L-lysine (molecular weight 4,000-15,000) were purchased from Sigma, St. Louis, Mo. Production of rabbit polyclonal antibodies 102c and 92e was reported previously (Grundke-Iqbal et al., 1988, Mol. Brain Res. 4:43-52; Iqbal et al., 1989, Proc. Natl. Acad. Sci. USA 86:5646-5650) Mouse monoclonal antibodies Tau-1 (Binder et al., 1985, J. Biol. Chem. 101:1371-1378), AT8 (Mercken et al., 1992, Neuropathol. 84:265-272), and PHF-1 (Greenberg et al., 1992, J. Biol. Chem. 267:564-569), were kindly provided by Drs. L. 1. Binder, University of Alabama, Birmingham, Ala.; A. Van de Voorde, Innogenetics, Industriepark Zwijinaarde, Belgium; and S. Greenberg, Burke Medical Research Institute, White Plains, N.Y., respectively. SMI33, SMI31, SMI34, goat anti-mouse IgG and peroxidase-anti-peroxidase complex were purchased from Sternberger Monoclonals Inc., Baltimore, Md. Alkaline phosphatase-conjugated goat anti-mouse and anti-rabbit IgG were purchased from Bio-Rad, Hercules, Calif.

Isolation of Tau

Abnormally phosphorylated Alzheimer tau (AD P-tau) and normal human tau were isolated by the method of Köpke et al. (1993, J. Biol. Chem. 268:24374-24384) from autopsied brains of a 70-year-old male with Alzheimer disease and a 51-year-old male normal case, respectively. Briefly, AD P-tau was isolated from a non-neurofibrillary tangle pool, the 27,000 g to 200,000 g fraction of the Alzheimer brain homogenate by extraction in 8 M urea, followed by dialysis against Tris buffer. This AD P-tau is readily soluble in buffer and abnormally phosphorylated as PHF-tau (Köpke et al., 1993, J. Biol. Chem. 268:24374-24384). Normal human tau was purified from 35-45% ammonium sulfate precipitates of the 200,000 g brain supernatant, followed by acid treatment (pH 2.7) and chromatography on a phosphocellulose column (Cellulose Phosphate P11, Whatman) (Köpke et al., 1993, J. Biol. Chem. 268:24374-24384).

Protein concentrations were determined by a modified Lowry assay (Bensadoun and Weinstein, 1976, Anal. Biochem. 70:241-250).

Preparation of [³²P]Substrates and Determination of Protein Phosphatase Activities

PP-2A and PP-2B activities were measured as described above by using [³²P]phosphorylase (0.9 mol ³²P incorporated/95,000 g) and [³²P]phosphorylase kinase (1.9 mol ³²P incorporated/335,000 g) as substrates, respectively (see Section 6). Phosphorylase and phosphorylase kinase phosphorylation were catalyzed by phosphorylase kinase and catalytic subunit of cAMP-dependent protein kinase, respectively. One unit of protein phosphatase activity is defined as that amount which catalyzes the release of 1.0 mmol phosphate per min from [³²P]substrate at 30° C.

Dephosphorylation of Abnormally Phosphorylated Alzheimzer Tau by PP-2A and PP-2B

Unless otherwise stated, dephosphorylation of AD P-tau by PP-2A, or PP-2A₂ was carried out at 30° C. in 50 mM Tris, pH 7.0, 20 mM β-mecaptoethanol, 0.1 mg/ml BSA, 1.0 mM MnCl₂, 50 μg/ml AD P-tau and 5.0 U/ml enzyme. In some experiments, Mn²⁺ in the reaction mixture was replaced by other effectors (see Figure legends). In the reaction mixture for PP-2B, MnCl₂ was substituted with-1.0 mM NiCl₂, 1.0 mM CaCl₂ and 1.0 μM calmodulin. The reaction was started by the addition of enzymes. After appropriate incubation times (see Figure legends), reactions were stopped by the addition of 5 volumes of cold acetone to precipitate proteins. Dephosphorylation was monitored by immunoblotting with the phosphorylation-dependent antibodies described below.

SDS-Polyacrylamide Gel Electrophoresis and Immunoblotting

The precipitated protein samples were dissolved in SDS-PAGE sample buffer and heated at 95° C. for 4 min, followed by 10% SDS-PAGE (1.0 μg tau protein/lane) as described by Laemmli (1970, Nature 227:680-685). Immunoblotting was carried out as described previously (Grundke-Iqbal et al., 1986, Proc. Natl. Acad. Sci. USA 83:4913-4917). The primary antibodies for immunoblotting and their epitopes have all been previously characterized. Antibodies 102c, Tau-1 and SMI33 recognize the dephosphorylated form of tau at Ser-46 (Iqbal et al., 1989, Proc. Natl. Acad. Sci. USA 86:5646-5650), Ser-199/Ser-202 (Biernat et al., 1992, EMBO J. 11:1593-1597; Grundke-Iqbal et al., 1986, Proc. Natl. Acad. Sci. USA 83:4913-4917; Szendrei et al., 1993, J. Neurosci. Res. 34:-243-249), and Ser-235 (Lichtenberg-Kraag et al., 1992, Proc. Natl. Acad. Sci. USA 89:5384-5388) respectively; whereas antibodies AT8, SMI31 and PHF-1 recognize tau phosphorylated at Ser-199/Ser-202 (Biernat et al., 1992, EMBO J. 11:1593-L597), Ser-396/Ser-404 (Lichtenberg-Kraaget al., 1992, Proc. Natl. Acad. Sci. USA 89:5384-5388) and Ser-396 (Greenberg et a., 1992, J. Biol. Chem. 267:564-569; Lang et al., 1992, Biochem. Biophys. Res. Commun. 187:783-790), respectively. These antibodies were used at dilutions of 1:100 for 102c, SMI31 and SMI34, 1:500 for SMI33 and PHF-1, 1:5,000 for 92e, and 1:500,000 for Tau-1. Antibody AT8 was used at a concentration of 2.0 μg/ml. The blots were developed by alkaline phosphatase staining (for 102c, Tau-1, SMI33, PHF-1 and 92e) or peroxidase staining (AT8, SMI31 and SMI34). The intensity of immunostaining of-some blots with Tau-1 was scanned by using a Shimadzu dual-wavelength flying-spot scanner.

### 7.2. Results

Comparison of Dephosphorylation of AD P-Tau by PP-2A₁, PP-2A₂ and PP-2B

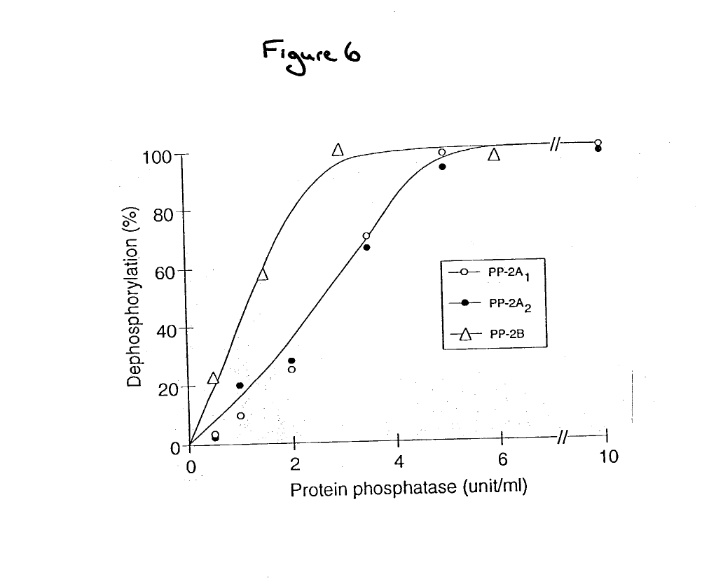

In a previous study, we found rapid dephosphorylation of AD P-tau by purified PP-2B but not by an equivalent amount of PP-2A (see Section 6). When incubated either for longer times at 30° C. or in the presence of Mn²⁺, PP-2A was also able to dephosphorylate AD P-tau at some of the abnormal phosphorylation sites. Therefore, in optimal conditions for both PP-2A and PP-2B, we have further compared the dephosphorylation of AD P-tau by different amounts of PP-2A (PP-2A, and PP-2A₂) and PP-2B (FIG. 6). Dephosphorylation of AD P-tau (Tau-1 epitope) was observed with a lower amount of PP-2B than PP-2A. Maximal dephosphorylation of AD P-tau was observed with about 3.0 U/ml PP-2B and about 5.0 U/ml of either PP-2A₁ or PP-2A₂. No significant differences in the dephosphorylation of AD P-tau between PP-2A₁ and PP-2A₂ were observed.

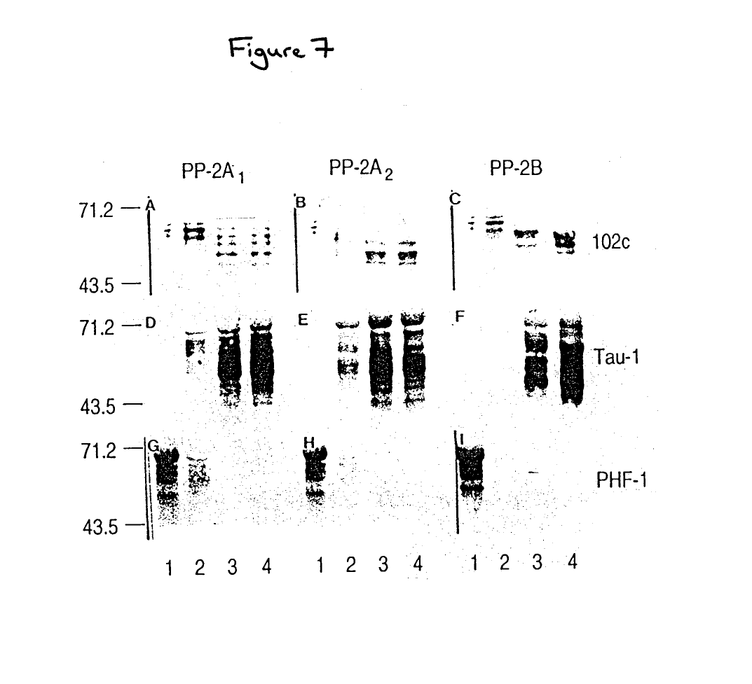

Dephosphorylation of AD P-tau by different amounts of PP-2A₁, PP-2A₂ and PP-2B was also determined by immunoblotting with antibodies 102c and PHF-1, which monitor the dephosphorylation at phosphorylation sites Ser46 and Ser-396 of the protein, respectively. The results indicated that, as with the Tau-1 sites, the dephosphorylation of the 12c and PHF-1 sites of AD P-tau required higher amounts of PP-2A, or PP-2A₂ than of PP-2B (FIG. 7).

Site-Specific Dephosphorylation of AD P-Tau by PP-2A₁ and PP-2A₂

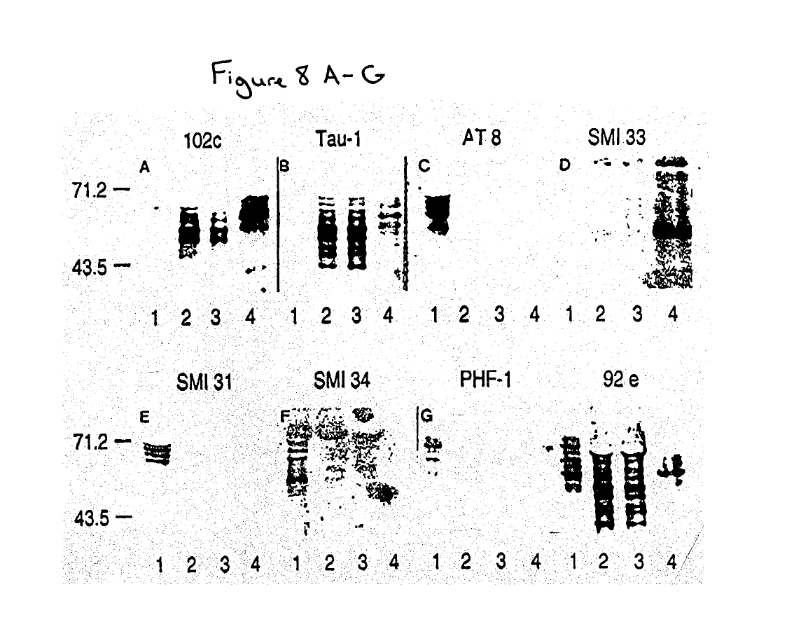

Site-specific dephosphorylation of AD P-tau by either 5.0 U/ml PP-2A, or 5.0 U/ml PP-2A₂ was studied. Eight antibodies, seven of which are phosphorylationdependent, were used for this experiment. Antibodies 102c, Tau-1 and SMI33 recognize the dephosphorylated form of tau at the sites Ser-46, Ser-199/Ser-202 and Ser-235, respectively. In contrast, antibodies AT8, SMI31 and PHF-1 recognize the phosphorylated form of tau at the sites Ser-199/Ser-202, Ser-396/Ser-404 and Ser-396, respectively. As shown in FIG. 8, dephosphorylation of AD P-tau either by PP-2A, or PP-2A, altered the accessibilities of the antibodies 102c, Tau-1, AT8, SMI31, SMI34 and PHF-1, but not of antibody SMI33This finding suggests that PP-2A₁ and PP-2A₂ can dephosphorylate AD P-tau at Ser-46, Ser-199, Ser-202, Ser-396 and Ser-404, but not at Ser-235. The two types of PP-2A, PP-2A, and PP-2A₂, showed almost no difference in dephosphorylation of AD P-tau (compare lanes 2 with 3 of panels A-G of FIG. 8).

Normal human tau has a higher relative mobility on SDS-PAGE than abnormally phosphorylated tau isolated from Alzheimer brain and tau phosphorylated by several protein kinases (Baudier and Cole, 1987, J. Biol. Chem. 262:17577-17583; Drewes et al., 1992, EMBO J. 11:2131-2138; Grundke-Iqbal et al., 1986, Proc. Natl. Acad. Sci. USA 83:49134917; Iqbal et al., 1986, Lancet 2:421426; Iqbal et al., 1989, Proc. Natl. Acad. Sci. USA 86:5646-5650; Mandelkow et al., 1992, FEBS Lett. 314:315-321; Vulliet et al., 1992, J. Biol. Chem. 267:22570-22574). The mobility shift of tau on SDS-PAGE has been used previously as a criterion of hyperphosphorylation of tau The seven phosphorylationdependent antibodies above can recognize either dephosphorylated (Tau-1 and SMI33) or untreated (AT8, SMI31, SMI34 and PHF-1) AD P-tau, except that 102c also weakly stains untreated AD P-tau We therefore used a phosphorylation-independent tau antibody, 92e, which recognizes both normal and AD P-tau, to monitor the mobility shift of AD P-tau after dephosphorylation by PP-2A (FIG. 8, H). This mobility shift was apparent after dephosphorylation both by PP-2A₁ and PP-2A₂ (FIG. 8, H). Similar results were also seen on immunoblots with antibody 102c (FIG. 8, A).

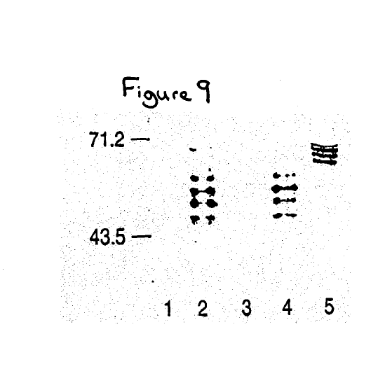

We have investigated the effects of a PP-2A inhibitor, okadaic acid, and protease inhibitors on the changes of relative electrophoretic mobility and of epitopes of above antibodies after treatment of AD P-tau with PP-2A As shown in FIG. 9, incubation of AD P-tau with PP-2A in the presence of 1.0 μM okadaic acid did not unmask Tau-1 epitope (lane 3), whereas both in the presence (lane 4) and the absence (lane 2) of protease inhibitor cocktail the Tau-1 epitope was unmasked. However, protease inhibitor cocktail could block the apace of the lowest faint band (compare lane 2 with lane 4), indicating that this band was contributed by proteolysis. Similar results were also observed by immunoblots with other antibodies (Figure not shown). This result confirmed that the epitope changes of AD P-tau resulted from dephosphorylation by PP-2A and the mobility shift of AD P-tau is mainly contributed by dephosphorylation.

Time Course of Dephosphorylation of AD P-tau by PP-2A₁ and PP-2A₂

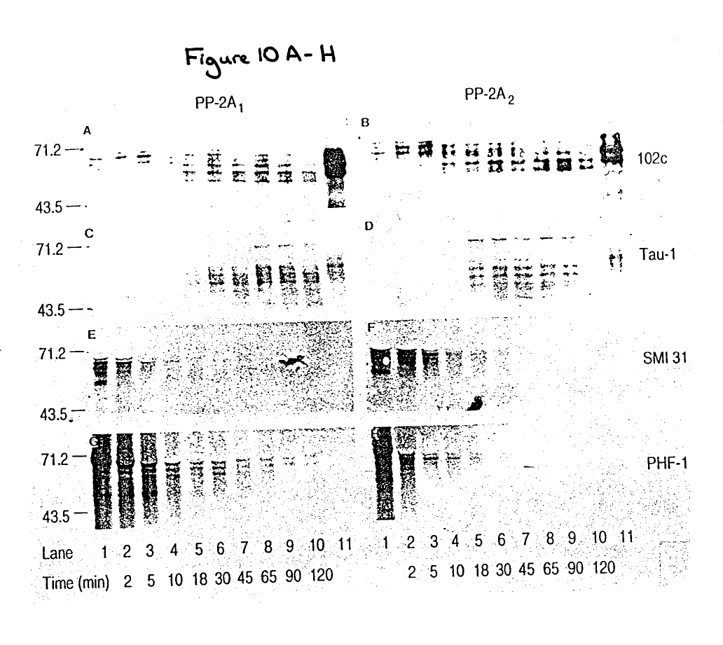

To observe the relative dephosphorylation rates of each specific site of AD P-tau by PP-2A₁ and PP-2A₂, we studied the time course of site-specific dephosphorylation of AD P-tau by these two types of PP-2A. The dephosphorylation was tested by antibodies 102c, Tau-1, SMI31 and PHF-1 (FIG. 10). Both PP-2A₁ and PP-2A₂ rapidly changed the epitopes of the four antibodies above. After 18-30 min, the change of staining density on blots was almost at its maximum. No significant difference of the dephosphorylation rates was observed when either PP-2A₁ or PP-2A₂ was used. These results indicate that the PP-2A isozymes have almost the same activity towards phosphorylated sites Ser46, Ser-199, Ser-202, Ser-396 and Ser-404 of AD P-tau. A mobility shift accompanying dephosphorylation was also clearly seen on immunoblots with antibody 102c (FIG. 10, A and B).

Effects of Mu²⁺, Mg²⁺ and Polylysine on Dephosphorylation of AD P-tau by PP-2A₁ and PP-2A₂

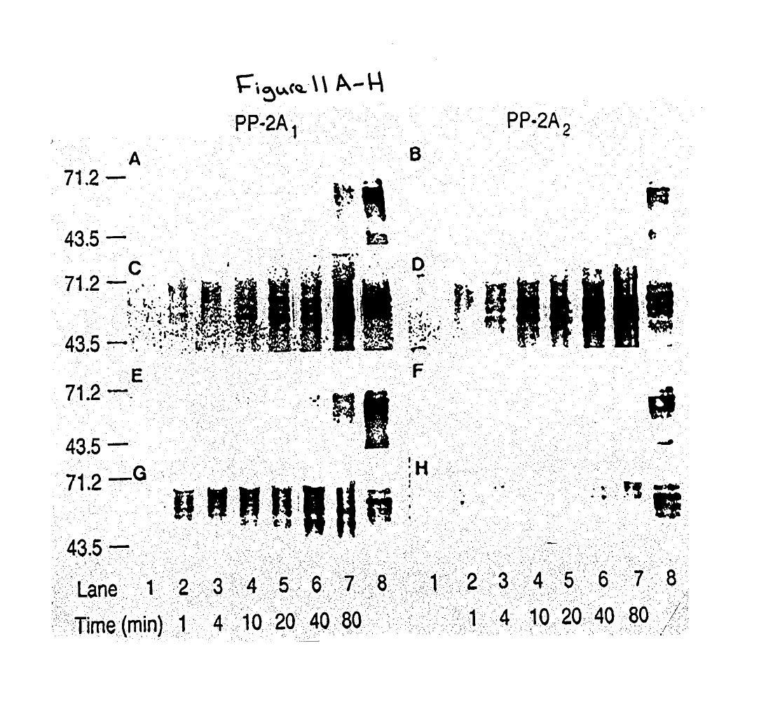

It is known that PP-2A activity is affected by divalent cations and basic proteins (Ballou and Fischer, 1987, The Enzymes 17:311-361; Cohen, 1989, Annu. Rev. Biochem. 58:453-508). Mn²⁺ and polylysine can activate PP-2A. These effects on PP-2A activity are variable, depending on the substrate used. In contrast, Mg²⁺ might slightly stimulate, inhibit or have no effect on PP-2A activity, also depending on the subtypes of the enzyme as well as the substrates used. We therefore investigated the effects of Mn²⁺, Mg²⁺ and polylysine on the dephosphorylation of AD P-tau by PP-2A, and PP-2A₂ (FIG. 11). In the absence of metals and polylysine (1.0 mM EDTA present), PP-2A₂ had very little activity towards AD P-tau (FIG. 11, B), whereas PP-2A₁ had some activity (FIG. 11, A). Mn²⁺ markedly stimulated the activities of both PP-2A₁ and PP-2A₂ (FIG. 11, compare C with A, and D with B). Polylysine also strongly activated PP-2A₁ (FIG. 11, compare G with A), but only slightly activated PP-2A₂ (FIG. 11, compare H with B and G). Mg²⁺ had no detectable effect on the activities of either PP-2A₁ or PP-2A₂ (FIG. 11, compare E with A, and F with B). In comparison, when [³²P]phosphorylase was used as a substrate, Mn²⁺ had almost no effect on PP-2A₁ activity, whereas polylysine slightly stimulated the activity; under similar conditions, Mn²⁺ stimulated PP-2A₂ much more strongly than polylysine did (data not shown). These results suggest that the modulation of AD P-tau phosphatase activity of PP-2A by Mn²⁺ and polylysine is different from that of other substrates.

### 7.3. Discussion

In the present study, we examined the dephosphorylation of AD P-tau by PP-2A₁ and PP-2A₂ in comparison with that by PP-2B. We found that both PP-2A and PP-2B could dephosphorylate AD P-tau, as determined by immunoblotting with phosphorylation-dependent anti-tau antibodies. Approximately twice as much PP-2A as PP-2B was needed for comparable dephosphorylation of AD P-tau.

PP-2A and PP-2B dephosphorylate different sites on AD P-tau. PP-2B can dephosphorylate AD P-tau at Ser46, Ser-199, Ser-202, Ser-235, Ser-396 and Ser-404 (see Section 6 hereinabove), whereas PP-2A failed to dephosphorylate Ser-235 of AD P-tau. Furthermore, the rate of dephosphorylation of different sites by PP-2A was almost the same, but that by PP-2B was nonidentical (see Section 6). Interestingly, Ser-235 was more rapidly dephosphorylated by PP-2B than other sites (see Section 6), whereas this site could not be dephosphorylated by PP-2A. These results suggest that probably both PP-2A and PP-2B are involved in the dephosphorylation of AD P-tau.

We have found that PP-2A can dephosphorylate AD P-tau at Ser-46, Ser-199, Ser-202, Ser-396 and Ser-404. Using [³²P]phosphorylase kinase as substrate, we previously observed that the PP-2A activity of Alzheimer disease brains was lower than that of age-matched controls (Gong et al., 1993, J. Neurochem. 61:921-927). Taken together, these results suggest that a deficiency of PP-2A might contribute to the abnormal hyperphosphorylation of tau in Alzheimer disease.

PP-2A may also be associated with tau phosphorylation indirectly. Recent studies have shown that PP-2A can inactivate mitogen-activated protein (MAP) kinase (Pelech and Sanghera, 1992, Science 257:1355-1356) and cdc2 (Clarke et al., 1993, Mol. Cell. Biol. 4:397-411). These two kinases have been reported to phosphorylate tau at some of the abnormally phosphorylated sites known to be present in AD P-tau (Drewes et al., 1992, EMBO J. 11:2131-2138; Vulliet et al., 1992, J. Biol. Chem. 267:22570-22574). Therefore, decreased PP-2A activity may result in MAP kinase and cdc2 kinase remaining in activated states for extended periods. Under these conditions, it is possible that tau may become hyperphosphorylated by these kinases.

PP-2A is present in vivo in two major forms, termed PP-2A₁ and PP-2A₂ (Cohen, 1989, Annu. Rev. Biochem. 58:453-508). PP-2A₁ generally has lower activity than PP-2A₂ a variety of substrates (Cohen, 1989, Annu. Rev. Biochem. 58:453-508), but this depends on the substrates used. Using phosphorylase as substrate to standardize PP-2A₁ and PP-2A₂ activities, we have found that they both had almost the same activities towards AD P-tau. The specificities for different sites on AD P-tau were also identical. Goedert et al. (1992, FEBS Lett. 312:95-99) have recently reported that the tau phosphatase:phosphorylase phosphatase activity ratio of PP-2A₁ was 7- to 8-fold higher than that of PP-2A₂. In their study, the tau phosphatase activity was determined by using [³²P]tau phosphorylated in Vitro by MAP kinase. Comparison of their results with ours indicates that AD P-tau and MAP kinase-phosphorylated tau are different substrates for PP-2A. In fact, not all abnormal sites of AD P-tau are phosphorylated by MAP kinase (Drewes et al., 1992, EMBO J. 11:2131-21383), and non-MAP kinase sites may alter the conformation of AD P-tau in such a way that it behaves as a different substrate.

In conclusion, PP-2A can dephosphotylate AD P-tau in vitro at some of the abnormal sites. PP-2A₁ and PP-2A₂ have almost the same activity towards AD P-tau. The dephosphorylation of AD P-tau by PP-2A is markedly stimulated by Mn²⁺. Regulation of tau dephosphorylation may be carried out by a combination of PP-2A and PP-2B. The deficiency of either. PP-2A or PP-2B, or both, might result in abnormal hyperphosphorylation of tau in Alzheimer disease brain.

### 8. EXAMPLE: DEPHOSPHORYLATION OF MICROTUBULE-ASSOCIATED PROTEIN TAU BY PROTEIN PHOSPHATASE-1 AND -2C AND ITS IMPLICATION IN ALZHEIMER DISEASE

As described herein, dephosphorylation of tau by protein phosphatase-1 and -2C was examined, using both AD P-tau and [³²P]tau labelled by cAMP-dependent protein kinase as substrates. Dephosphorylation of AD P-tau was monitored by its interaction with the following phosphorylation-dependent antibodies: 102c, Tau-1, SMI33, SMI31 and PHF-1. The abnormally phosphorylated sites Ser-199, Ser-202, Ser-396 and Ser-404 but not Ser-46 and Ser-235 of AD P-tau (the amino acids are numbered according to the largest isoform of human tau, tau₄₄₁) were found to be dephosphorylated by protein phosphatase-1 and this dephosphorylation was activated by Mn²⁺. In contrast, protein phosphatase-2C did not dephosphorylate any of these sites. Both protein phosphatase-1 and -2C had high activities towards [³²P]tau phosphorylated by cAMP-dependent protein kinase. These results suggest that both protein phosphatase-1 and -2C might be associated with the normal phosphorylation state of tau, but only the former and not the latter phosphatase is involved in its abnormal phosphorylation in Alzheimer disease

### 8.1. Materials and Methods

Materials

Phosphorylase kinase was purified from rabbit skeletal muscle by the method of Cohen (1973, Eur. J. Biochem. 34:1-14) cAMP-dependent protein kinase (PKA) was purchased from Sigma, St. Louis, Mo., USA. Rabbit skeletal muscle PP-1 was purchased from Upstate Biotechnology Inc., Lake Placid, N.Y. PP-2B (holoenzyme) was purified from bovine brain according to the method of Sharma et al. (1983, Meth. Enzymol. 102:210-219). PP-2C was purified from bovine kidney as previously described (Amick et al., 1992, Biochem. J. 287:1019-1022).

Polyclonal antibodies 102c were raised as previously reported (Iqbal et al., 1989, Proc. Natl. Acad. Sci. USA 86:5646-5650). Monoclonal antibodies Tau-1 and PHF-1 were kindly provided by Drs. L. I. Binder (Binder et ad., 1985, J. Cell Biol. 101, 1371-1378) and S. Greenberg (Greenberg et al., 1992, J. Biol. Chem. 267:564-569), respectively; SMI33, SMI31, goat anti-mouse IgG and peroxidase-anti-peroxidase complex were purchased from Sternberger Monoclonals Inc., Baltimore, Md. Alkaline phosphatase-conjugated goat anti-mouse and anti-rabbit IgG were purchased from Bio-Rad, Hercules, Calif.

Isolation of Tau

AD P-tau and normal human tau were isolated from autopsied brains of a 70-year-old male with Alzheimer disease and a 51-year-old male normal case, respectively (Köpke et al., 1993, J. Biol. Chem. 268:24374-24384). Briefly, AD P-tau was isolated from a non-neurofibrillary tangle pool, the 27,000 g to 200,000 g fraction of the Alzheimer brain homogenate was extracted in 8 M urea, followed by dialysis against Tris buffer. This AD P-tau is readily soluble in buffer and abnormally phosphorylated as PHF-tau (Köpke et al., 1993, J. Biol. Chem. 268:24374-24384). Normal human tau was purified according to Köpke et al. (1993, 3. Biol. Chem. 268:24374-24384) from the 35-45% ammonium sulfate precipitates of 200,000 g brain supernatant, followed by acid treatment (pH 2.7) and chromatography on a phosphocellulose column (Cellulose Phosphate P11, Whatman).

Protein concentrations were determined by a modified Lowry assay (Bensadoun and Weinstein, 1976, Anal. Biochem. 70, 241-250).

Preparation of [3P]Phosphorylase Kinase and Determination of Protein Phosphatase Activities

[³²P]phosphorylase kinase (1.9 mol ³²P incorporated/335,000 g) phosphorylated by catalytic subunit of cAMP-dependent protein kinase was prepared as reported previously (Gong et al., 1993, J. Neurochem. 61:921-927). The activities of PP-1, PP-2B and PP-2C were measured by counting the radioactivity released from [³²P]substrate as previously described (Gong et al., 1993, J. Neurochem. 61:921-927). The reaction mixtures contained 50 mM Tris, pH 7.0, 20 mM β-mecaptoethanol, 1.0 mM MnCl₂ and 1.0 μM [³²P]phosphorylase kinase for PP-1. For PP-2B and PP-2C activities, MnCl₂ was substituted by 1.0 mM CaCl₂ and 10 μM calmodulin, and 10 mM MgCl₂, respectively. One unit of protein phosphatase activity is defined as that amount which catalyzes the release of 1.0 mmol phosphate per min from [³²P]phosphorylase kinase at 30° C.

Treatment of AD P-Tau with Protein Phosphatases

Unless otherwise stated, dephosphorylation of AD P-tau was carried out at 30° C. in 50 mM Tris, pH 7.0, 10 mM β-mecaptoethanol, 0.1 mg/ml BSA, 50 μg/ml AD P-tau and PP-1, PP-2B or PP-2C. In some experiments, several effectors were added in the reaction mixture (see Results section). The reaction was started by addition of the enzyme. After appropriate incubation times (see Figure legends), reactions were stopped by addition of 5 volumes of cold acetone to precipitate proteins. The precipitated protein samples were dissolved in SDS-PAGE sample buffer (60 mM Tris-HCl, pH 68, 3% SDS, 5% β-mercaptoethanol, 10% glycerol and 0.05% bromophenol blue) and heated at 95° C. for 4 min, followed by 10% SDS-PAGE. Immunoblotting was carried out as described previously (Grundke-Iqbal et al., 1986, Proc. Natl. Acad. Sci USA 83:4913-4917). The primary antibodies for immunoblotting and their epitopes have all been previously characterized. They are phosphorylationdependent as well as site-specific. Briefly, antibodies 102c (Iqbal et al., 1989, Proc. Natl. Acad. Sci. USA 86:5646-5650), Tau-1 (Grundke-Iqbal et al., 1986, Proc. Natl. Acad. Sci. USA 83:4913-4917; Biernat et al., 1992, EMBO J. 11, 1593-1597; Szendrei et al., 1993, J. Neurosci. Res. 34:243-249) and SMI33 (Lichtenberg-Kraag et al., 1992, Proc. Natl. Acad. Sci. USA 89:5384-5388) recognize dephosphorylated form of tau at sites Ser-46, Ser-199/Ser-202 and Ser-235, respectively. Antibodies SMI31 (Lichtenberg-Kraag et al., 1992, Proc. Natl. Acad. Sci. USA 89:5384-5388) and PHF-1 (Lang et al., 1992, Biochem. Biophys. Res. Commun. 187:783-790) recognize tau phosphorylated at Ser-396/Ser-404 and Ser-396, respectively. These antibodies were used at dilutions of 0.4 μg/ml for 102c, 1:100 for SMI31, 1:500 for SMI33 and PHF-1, and 1:500,000 for Tau-1.

Preparation of [³²P]Tau and Dephosphorylation of [³²P]Tau by Protein Phosphatases

Tau purified from normal human brain was phosphorylated with [³²P]ATP by PKA as described by Scott et al. (1993, J. Biol. Chem. 268:1166-1173). About 2 mol ³²P/mol tau was incorporated by PKA. Dephosphorylation of [³²P]tau by PP-1, PP-2A and PP-2B was carried out employing the same conditions as when AD P-tau was used as a substrate. The phosphatase activities were measured by counting the radioactivity released from [³²P]tau as previously described (Gong et al., 1993, J. Neurochem. 61:921-927).

### 8.2. Results

Definition of PP-1 and PP-2C Activities and their Modulation by MD²⁺ and Mg²⁺

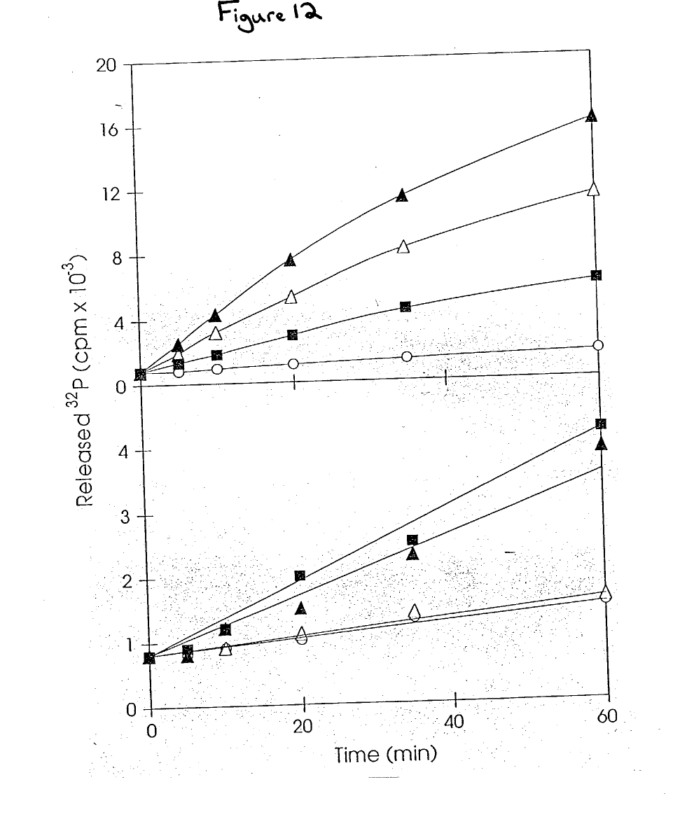

To observe and compare the potential dephosphorylation of AD P-tau by various protein phosphatases, the same amount of enzyme activity of each phosphatase should be used. Hence we employed [³²P]phosphorylase kinase as a substrate to standardize the activities of PP-1 and PP-2C. PP-1 and PP-2C activities are modulated by cations and each enzyme preparation responds differently to divalent cations (Ballou and Fisher, 1987, The Enzymes 17:311-361). We therefore determined PP-1 and PP-2C activities in the absence and presence of either Mn²⁺ or Mg²⁺ using [³²P]phosphorylase kinase as a substrate. As shown in FIG. 12, PP-1 was activated by 1.0 mM Mn²⁺ but inhibited by. 10 mM Mg²⁺. PP-2C was Mg²⁺- or Mn²⁺-dependent, and no activity was detected in the absence of Mg²⁺ or Mn²⁺. Highest activities were obtained using 1.0 mM Mn²⁺ for PP-1 and 10 mM Mg²⁺ for PP-2C. Hence these conditions were used to study the in vitro dephosphorylation of AD P-tau and PKA-phosphorylated tau.

Treatment of AD P-Tau with PP-1 and PP-2C

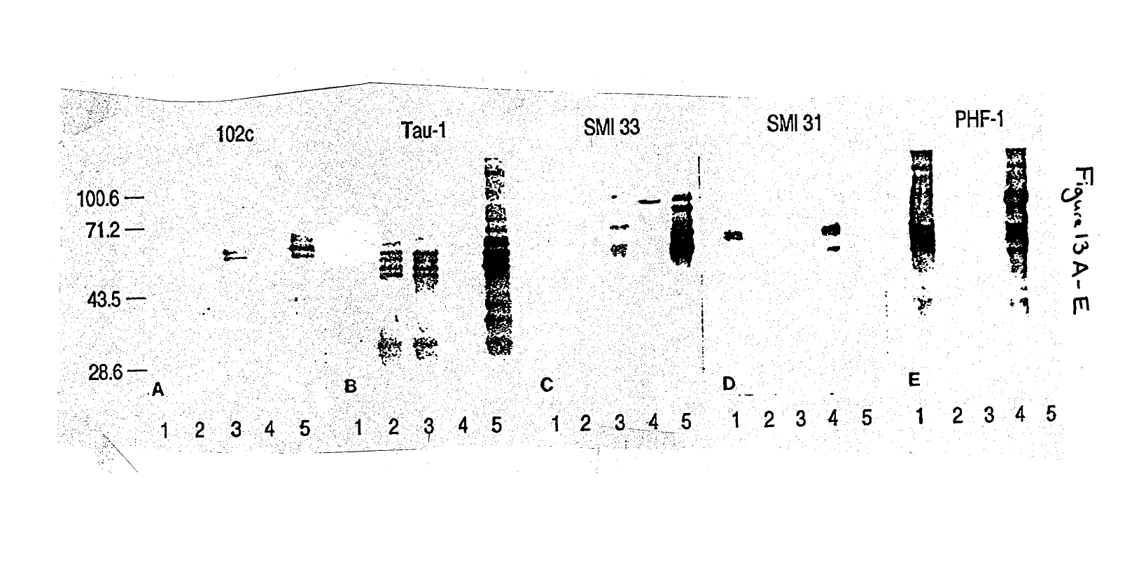

We have previously shown that PP-2A dephosphorylated abnormal phosphorylation sites Ser-46, Ser-199, Ser-202, Ser-396 and Ser-404 of AD P-tau (see supra), and that in addition to above sites, PP-2B also dephosphorylated another abnormal phosphorylation site, Ser-235 (see supra) In the present study, using immunoblots with five site-specific phosphorylation-dependent antibodies which recognize six abnormal phosphorylation sites of AD P-tau, we have further examined whether PP-1 and PP-2C can also dephosphorylate these abnormal phosphorylation sites at optimum in vitro conditions. PP-2B was employed as a positive control. We found that PP-1 unmasked the epitope of antibody Tau-1 and blocked the epitopes of antibodies SMI31 and PHF-1, but failed to unblock the epitopes of antibodies 102c and SMI33 (FIG. 13) PP-2C did not change any of these epitopes These results indicate that PP-1 dephosphorylates abnormal phosphorylation sites Ser-199, Ser-202, Ser-396 and Ser-404 but not Ser-46 and Ser-235 of AD P-tau. Whereas PP-2C had no effect on dephosphorylation of above sites.

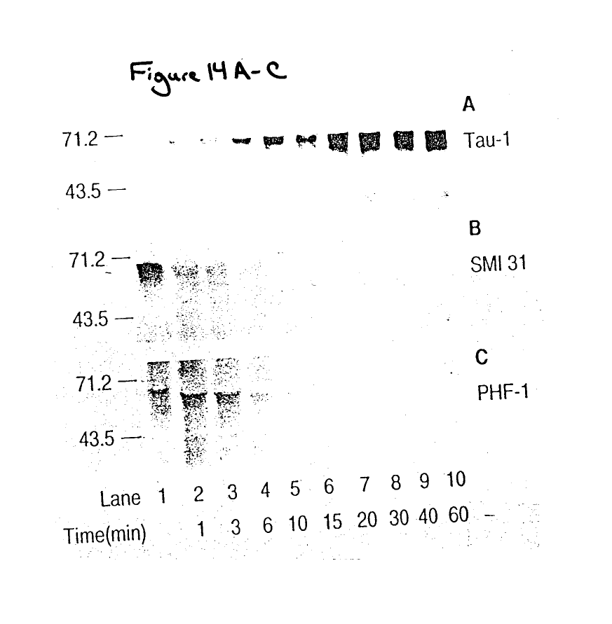

The rate of dephosphorylation of Ser-199/Ser-202, Ser-396/Ser-404 and Ser-396 of AD P-tau by PP-1 was determined using immunoblots with Tau-1, SMI31 and PHF-1, respectively. The time course showed a rapid change of epitopes of AD P-tau towards these three antibodies (FIG. 14). Within 20-30 min incubation of AD P-tau with PP-1, staining of Tau-1 became maximal and those of both PHF-1 and SMI31 disappeared completely. These results suggested that the four phosphorylation sites of AD P-tau can be easily hydrolyzed by PP-1.

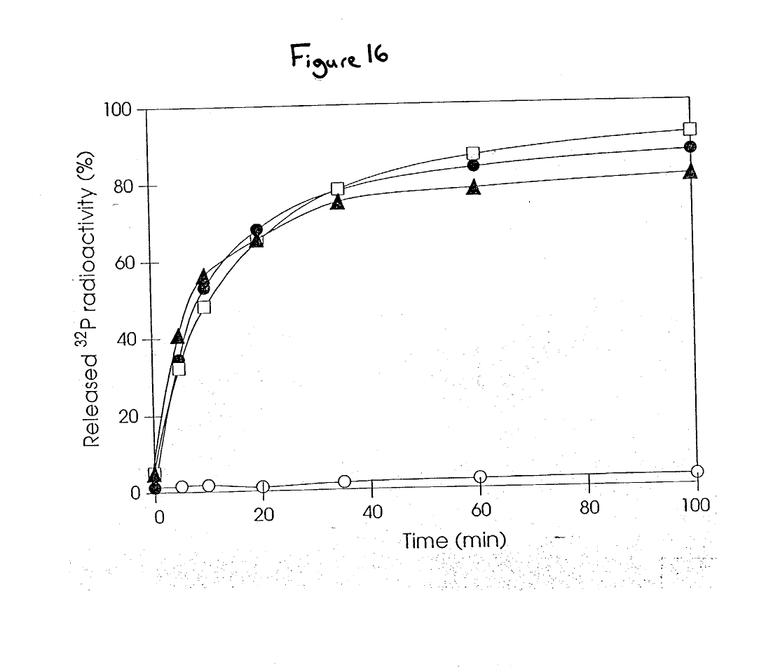

We have also investigated the dephosphorylation of AD P-tau at various conditions (FIG. 15). In the absence of metal (1.0 mM EDTA present), PP-1 could also dephosphorylate AD P-tau at Ser-396 but the activity was low. Dephosphorylation of AD P-tau by PP-1 was strongly activated by 1.0 mM Mn²⁺ but inhibited by 10 mM Mg²⁺. We further investigated the required concentration of Mn²⁺ for this activation. The activation was observed at 10 μM Mn²⁺ and it reached maximum at about 100 μM Mn²⁺ (data not shown).

Dephosphorylation of [³²P]tau by PP-1 and PP-2C

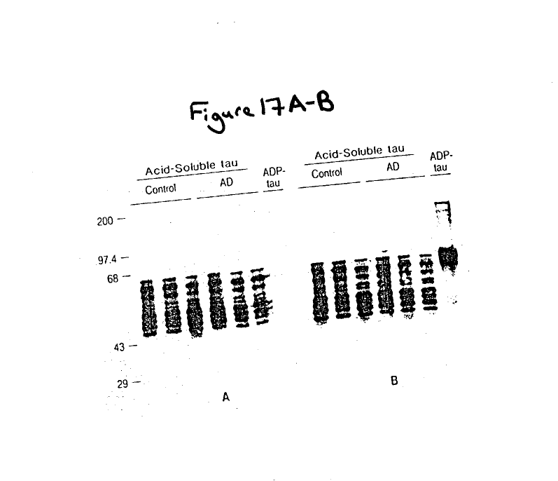

Tau protein is known to be phosphorylated in vitro at Ser-214, Ser-324, Ser-356, Ser-409 and Ser-416 by PKA (Scott et al., 1993, J. Biol. Chem. 268:1166-1173). So far none of these sites have been reported to be abnormally phosphorylated in Alzheimer disease brain, but they may be involved in normal phosphorylation of tau. We therefore asked whether these non-abnormal phosphorylation sites of tau can be dephosphorylated by either PP-1 and PP-2C. Interestingly, even though PP-1, PP-2B and PP-2C had obviously different effects on dephosphorylation of abnormal phosphorylation sites of AD P-tau, they had similar high activities towards [³²P]tau phosphorylated by PKA (FIG. 16).

### 8.3. Discussion

We have found that PP-2A and PP-2B rapidly dephosphorylated AD P-tau in vitro (Sections 6, 7 supra). The present study shows that PP-1 also dephosphorylates AD P-tau at some of the sites whereas PP-2C has no activity towards any of the sites studied. Although three of four types of protein phosphateses can dephosphorylate AD P-tau, they have different site specificities. Six of nine known abnormal phosphorylation sites of AD P-tau have been examined in these studies. They are Ser-46, Ser-199, -Ser-202, Ser-235, Ser-396 and Ser-404. All of them can be dephosphorylated by PP-2B; PP-2A can dephosphorylate all except S-235; and PP-1 dephosphorylates Ser-199, Ser-202, Ser-396 and Ser-404 but neither Ser-46 nor Ser-235. Hence at least four abnormal phosphorylation sites, Ser-199, Ser-202, Ser-396 and Ser-404, can be dephosphorylated by the three enzymes, PP-1, PP-2A and PP-2B. These results indicate that the regulation of phosphorylation level of tau is very complex and more than one protein phosphatase might be involved in hyperphosphorylation of tau in AD.

When [³²P]phosphorylase kinase was used as a substrate to determine protein phosphatase activities, PP-1 activity was found about 20-fold higher than PP-2C activity in human brain extracts (Gong et al., 1993, J. Neurochem. 61:921-927). In this study, also using [³²P]phosphorylase kinase as a substrate to define the activities of both PP-1 and PP-2C, 1.0 unit/ml of PP-1 almost completely dephosphorylated Ser-199, Ser-202, Ser-396 and Ser-404 of AD P-tau in 20 min, but 2.0 units/ml of PP-2C did not dephosphorylate AD P-tau at any sites studied at the optimal in vitro conditions. Taken together, these results suggest that AD P-tau is not a substrate for PP-2C.

Tau isolated from adult brain normally contains 2-3 moles-of phosphate per mole of the protein (Selden and Pollard, 1983, J. Biol. Chem. 258:7064-7071; Ksiezak-Reding et al., 1992, Brain Res. 597:209-219; Köpke et al., 1993, J. Biol. Chem. 268:24374-24384). However, neither the phosphorylation sites nor the responsive kinase(s) have yet been fully elucidated. Normal tau might be partially phosphorylated at Ser-202 and Ser-404 (Arioka et al., 1993, J. Neurochem. 60:461468; Poulter et al., 1993, J. Biol. Chem. 268:9636-9644), but to date other sites have not been excluded to be phosphorylated. PKA was known to phosphorylate tau at Ser-214, Ser-234, Ser-356, Ser409 and Ser416 (Scott et al., 1993, J. Biol. Chem. 268:1166-1173). PP-2C as well as PP-1 and PP-2B can release about 80% radioactivity from PKA-phosphorylated [³²P]tau in 60 min, suggesting that these phosphatases dephosphorylate most of these phosphorylation sites of tau. Therefore, even if it is not involved in abnormal phosphorylation of AD P-tau, PP-2C may be associated with the regulation of phosphorylation level of normal tau.

The present study also shows that PKA-phosphorylated tau and AD P-tau serve as different substrates for protein phosphatases. AD P-tau can be dephosphorylated by PP-1, PP-2A and PP-2B but not by PP-2C, whereas PKA-phosphorylated tau is almost an equally good substrate for PP-1, PP-2B, and PP-2C. The completely different behavior of AD P-tau and PKA-phosphorylated tau as a substrate for PP-2C may be due to different phosphorylation sites and/or due to different protein conformations of the in vitro-phosphorylated vs. the pathological AD P-tau. The pathological conformation of AD P-tau might make tau easily polymerize into PHF in Alzheimer brain. The results of this study also suggest that caution ought to be used when interpreting data obtained employing [³²P]tau as a model substrate to study potential protein phosphatase(s) involved in the pathogenesis of AD. This is important, especially because so far no protein kinase including mitogen-activated protein kinase has been reported to phosphorylate tau at all the known abnormal phosphorylation sites. These different phosphorylation sites between ³²P-labelled tau and AD P-tau could result in different conformations so that ³²P-labelled tau and AD P-tau serve as different substrates for protein phosphatases.

### 9. EXAMPLE: ROLE OF ABNORMALLY PHOSPHORYLATED TAU IN THE BREAKDOWN OF MICROTUBULES IN ALZHEIMER DISEASE

As described herein, to understand the role of the abnormal phosphorylation of tau in microtubule disruption in AD brain, we studied the ability of the normal cytosolic, and the AD P-tau to bind to tubulin and to promote microtubule assembly and investigated the effect of alkaline phosphatase treatment of tau on microtubule assembly. Tau isolated from a 2.5% perchloric extract of AD brain had almost the same activity as that obtained from control brain, and this activity did not change significantly on dephosphorylation Abnormally phosphorylated tau (AD P-tau) isolated from brain homogenate of AD cases had little activity, and on dephosphorylation with alkaline phosphatase, its activity increased to approximately the same level as the acid-soluble tau. Addition of AD P-tau to a mixture of normal tau and tubulin inhibited microtubule assembly. AD P-tau bound to normal tau but not to tubulin. These studies suggest that the abnormal phosphorylation of tau might be responsible for the breakdown of microtubule in affected neurons in AD not only because the altered protein has little microtubule-promoting activity, but also because it interacts with normal tau, making the latter unavailable for promoting the assembly of tubulin into microtubules.

### 9.1. Materials And Methods

Tissue Source and Preparation of Brain Cytosol. Six brains with histopathologically confirmed AD diagnosis and, as a control, six Huntington disease brains obtained between 3 to 5 h postmortem and stored frozen at −75° C. were used. Cytosol was obtained by centrifugation (100,000×g for 1 h) of frontal cortex homogenate (1 g/0.5 ml) in microtubule assembly buffer (see below) containing protease inhibitors (Köpke et al., 1993, J. Biol. Chem. 268:24374-24384).

Antibodies. Monoclonal antibody (mAb) Tau-1, ascites (Binder et al., 1985, J. Cell Biol. 101:1371-1378), and antiserum 92e to bovine tau C(Grundke-Iqbal et al., 1988, J. Mol. Brain Res. 4:43-52) were used at a dilution of 1:25,000 and 1:5,000, respectively; mAb DM1-A (Sigma, St. Louis, Mo.) against tubulin was used at a 1:1,000 dilution.

Isolation of AD P-tau and Acid-soluble Tau. AD P-tau was isolated by the method of Köpke et al. (1993, J. Biol. Chem. 268:24374-24384). For acid-soluble tau AD and control brains were homogenized with an Omnimixer at 4° C. in 2% perchloric acid (10 ml/g of tissue) containing protease inhibitors, as described previously (Köpke et al., 1993, J. Biol. Chem. 268:24374-24384). The homogenates were spun at 100,000×g for 30 min. The supernatant was brought to 2.5% perchloric acid and centrifuged again for another 30 min. The supernatant was concentrated approximately 10 times by Amicon filtration and dialyzed against 20 mM sodium acetate buffer, pH 5.6. After dialysis, the extract was spun for 10 min at 100,000×g, and the supernatant was subjected to carboxyl methyl chromatography using Millipore Mem Sep CM 1010 disk (Millipore, Bedford, Mass.). The protein sample (25-40 mg/50 ml) was loaded at a flow rate of 0.5 ml/min, and tau was eluted with 0.25 M NaCl in 20 mM sodium acetate buffer, pH 5.6. The eluate was analyzed by absorbance at 254 nm and immunoslot blot using antiserum 92e to tau. The tau peak was pooled and dialyzed against 5 mM MES buffer, pH 6.7, containing 0.05 mM EGTA. Aliquots of approximately 500 μl, containing 120 μg of protein, were dried in a Speed Vac concentrator (Savant, Farmingdale, N.Y.). For each assay, the lyophilized tau preparation was reconstituted in 1/10 vol. water immediately before use.

Protein Determination, Immunoblots, Radioimmuno-slot-blot, and Dephosphorylation. Protein concentrations were estimated by the method of Bensadoun and Weinstein (Bensadoun and Weinstein, 1976, Anal. Biochem. 70:241-250). Sample preparation and immunoblots were carried out as described previously (Grundke-Iqbal et al, 1984, Acta Neuropathol (Berl.) 62:259-267). The levels of normal and AD P-tau were determined by the radioimmuno-slot-blot method of Khatoon et al. (1992, J. Neurochem. 59:750-753). To detect AD P-tau, the blots were pretreated with alkaline phosphatase, 86 μg/ml in 0.1 M Tris, pH 8.0, and 1 mM phenylmethylsulfonyl fluoride for 15 h prior to immunostaining with mAb Tau-1.

Isolation of Tubulin. Rat brain tubulin was isolated through two temperature-dependent cycles of microtubule polymerization-depolymerization (Shelanski et al., 1973, Proc. Natl. Acad. Sci. USA 70:765-768) followed by phosphocellulose ion-exchange column chromatography (Sloboda and Rosenbaum, 1979, Biochemistry 18:48-55).

Microtubule Assembly-disassembly Assay. Tau (0.1-0.4 mg/ml) was mixed at 4° C. with purified rat brain tubulin (2 mg/ml) and 1 mM GTP, all in polymerization buffer (100 mM MES, pH 6.7, 1 mM EGTA and 1 mM MgCl₂). Tau was added last to initiate the reaction. After rapid mixing, the samples were pipetted into quartz microcuvettes and equilibrated at 37° C. in a thermostatically-controlled Cary 1 recording spectrophotometer. Solution turbidity was continuously monitored at 350 nm Steady state values were determined by measuring the total absorbance change after a turbidity plateau was reached. For the disassembly assay, after the steady state was reached, the reaction mixture was cooled to 6° C., and the turbidity was monitored. The state of assembly of microtubules was confirmed by negative stain electron microscopy (Wisniewski et al., 1984, J. Neuropathol. Exper. Neurol. 43:643-656).

In vitro dephosphorylation of tau was carried out by using the following conditions: acid-soluble tau from AD and control brains, and AD P-tau from AD brains were reconstituted with water to a final protein concentration of 0.1-0.2 mg/ml and then dialyzed against 0.1 M Tris, pH 8.0, containing 1 mM phenylmethylsulfonyl fluoride. The dialyzed samples were treated with alkaline phosphatase (500 Units/ml) and a mixture of protease inhibitors (10 μM leupeptin, 0.31 μM aprotinin, and 1.46 μM pepstatin) at 37° C. for 15 h. After this incubation, samples were dialyzed against 5 mM MES, pH 6.7, containing 0.05 mM E6TA, boiled for 5 min to inactivate the alkaline phosphatase, and then centrifuged at 15,000×g for 10 min. The phosphatase-control samples were treated identically, except that alkaline phosphatase was omitted and the samples were kept at 4° C. Unless otherwise stated, all steps were carried out at 4° C.

Dot Overlay Assay. AD P-tau interaction was carried out as described by Kremer et al. (1988, Anal. Biochem. 175:91-95). Different amounts (0.5, 1, 2 and 3 μg) of AD P-tau were dotted on nitrocellulose paper and overlaid with either normal human tau (8 μg/ml) or tubulin (10 μg/ml). All the incubations, blocking, and washing were done as previously described (Kremer et al., 1988, Anal. Biochem. 175:91-95). Tau was detected by using Tau-1 antibody, whereas DM1-A antibody was used for tubulin.

### 9.2. Results

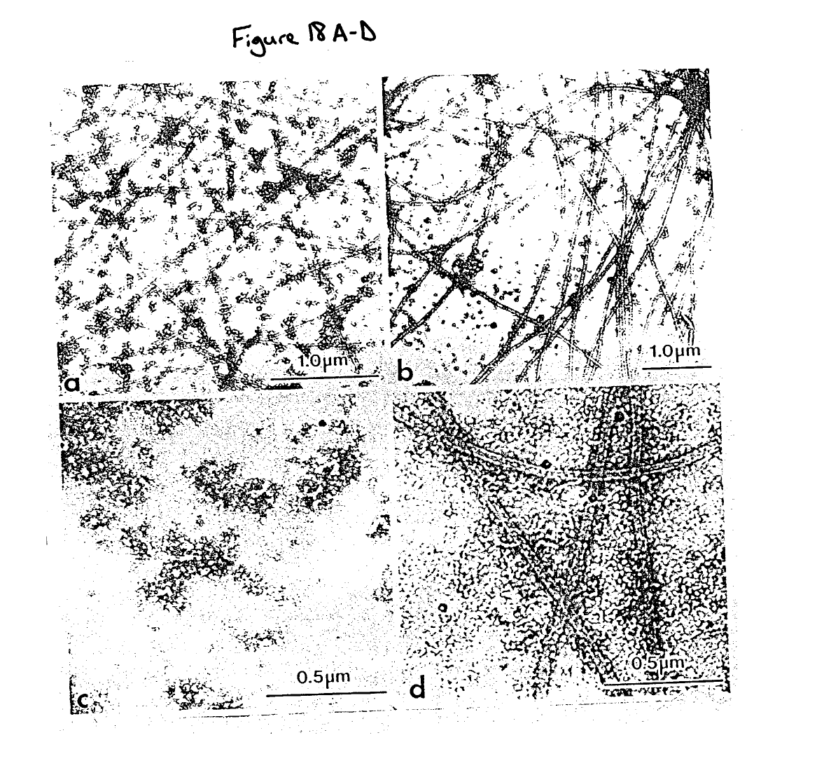

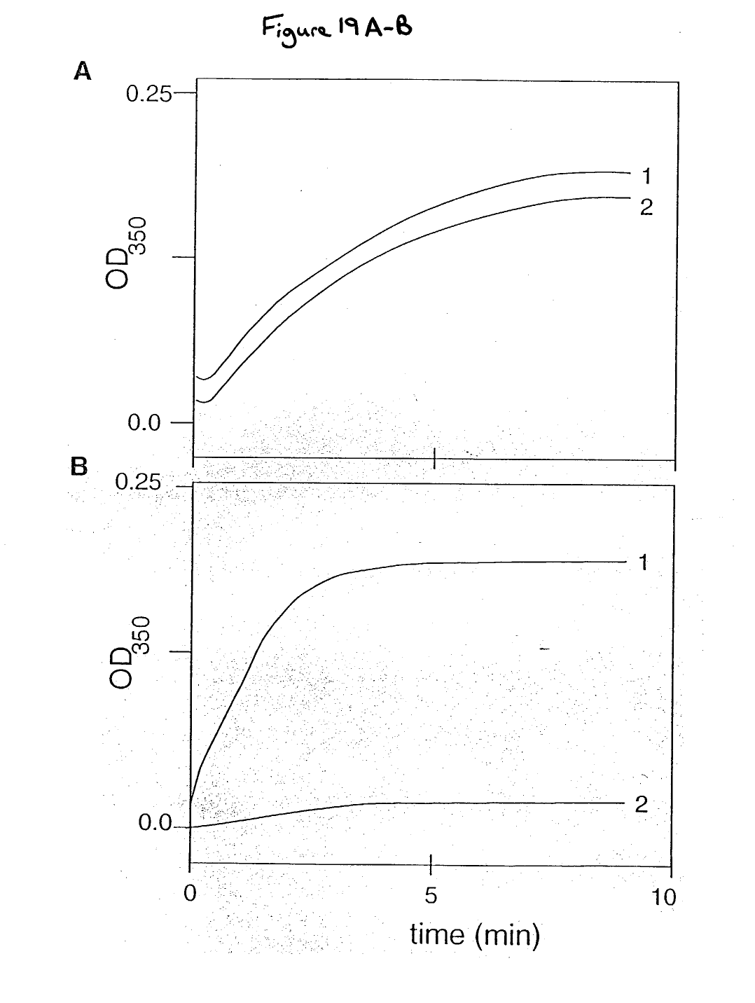

Microtubule Assembly-promoting Activity of 2.5% Perchloric Acid-Soluble Tau From AD and Control Brains Is Similar. Acid-soluble tau was isolated from 6 AD and 6 control brains. No AD P-tau was detected, and the pattern of tau isotypes was very similar in all the preparations (FIG. 17), although the yield of tau from AD brains was approximately 30% lower than that from the control brains (AD brains: 0.020±0.004 mg/g of tissue; control brains: 0.029±0.002 mg/g of tissue). The total protein composition of the tau preparations was also very similar (data not shown). The microtubule assembly-promoting activity of AD acid-soluble tau was not significantly different from that of control tau, as determined by the amount of microtubules formed at steady state and the rates of assembly and disassembly (Table 4). Furthermore, no ultrastructural differences, either in length or appearance, were detected between the microtubules obtained with the two tau preparations (FIGS. 18a and b).

TABLE 4*
Microtubule Assembly-Promoting and
Stabilizing Activities of Cytosolic
Acid-Soluble Tau from AD and Control Brain

| | Alzheimer | Control |
|---|---|---|
| Assembly (%) | 97 ± 14 (6)^{a} | 100 ± 16 (6) |
| Rate of assembly (min⁻¹) | 0.10 ± 0.02 (5) | 0.10 ± 0.02 (6) |
| Rate of disassembly (min⁻¹) | 0.19 ± 0.06 (5) | 0.15 ± 0.03 (6) |
| *The assembly assay was performed using fresh rat tubulin and AD or control acid-soluble tau. Values of OD (0.100 to 0.400) at steady state of polymerization were normalized by using those obtained with the control preparations as 100%. The rate of assembly was calculated from the initial slope. For the rate of disassembly, the temperature was set at 6° C., | | |
| #and the turbidimetric changes were recorded at 350 nm. The rate was calculated from the slope if disassembly. Both rates were determined by using the same amount of tau. | | |

^{a}Mean ± SD of cases studied in four different experiments carried out with three different concentrations of tau (0.1, 0.2, and 0.4 mg/ml). None of the differences between AD and control groups were statistically significant.

The assembly assay was performed using fresh rat tubulin and AD or control acid-soluble tau. Values of OD (0.100 to 0.400) at steady state of polymerization were normalized by using those obtained with the control preparations as 100% The rate of assembly was calculated from the initial slope. For the rate of disassembly, the temperature was set at 6° C., and the turbidimetric changes were recorded at 350 nm. The rate was calculated from the slope of disassembly. Both rates were determined by using the same amount of tau. ^{a}Mean+SD of cases studied in four different experiments carried out with three different concentrations of tau (0.1, 0.2, and 0.4 mg/ml). None of the differences between AD and control groups were statistically significant.

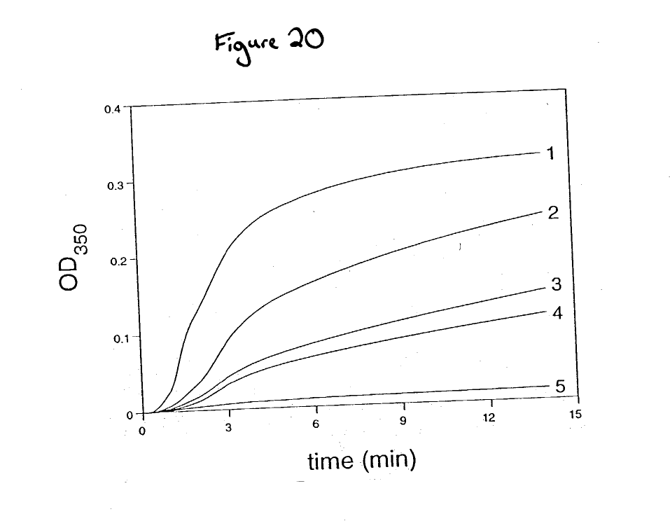

Dephosphorylation Increases the Microtubule Assembly-promoting Activity of AD P-tau but Not That of AD Acid-Soluble Tau. In vitro phosphorylation of tau diminishes its ability to promote the assembly of tubulin into microtubules (Lindwall and Cole, 1984, J. Biol. Chem. 259:5301-5305). Experiments were performed to determine whether dephosphorylation of AD acid-soluble tau and AD P-tau affects this property. The amount of the AD P-tau in the acid-soluble preparations was undetectable, as judged by the increase of Tau-1 immunoreactivity after dephosphorylation on Western blots (FIG. 17) and by immuno-slot-blot assay (data not shown). In contrast, the AD P-tau was labeled intensely with Tau-1 on immunoblots treated with alkaline phosphatase and was hardly detectable before dephosphorylation (FIG. 17). The dephosphorylation treatment had no effect on the microtubule assembly-promoting activity of AD acid-soluble tau, whereas it increased markedly the activity of AD P-tau, bringing it to approximately the same level as that obtained with the acid-soluble tau (FIG. 19). Before alkaline phosphatase treatment, only an occasional microtubule could be seen by electron microscopy (FIG. 18c). After the alkaline phosphatase treatment, many microtubules with no ultrastructural differences from those formed with AD acid-soluble tau were observed (FIG. 18d).

AD Cytosolic Fraction Is Able to Promote Microtubule Assembly. The effect of dephosphorylation of tau on microtubule assembly was also studied in brain cytosol. The concentration of normal tau in AD brain cytosols was approximately 65% of the corresponding value in the control cases (Table 5).

TABLE 5
Tau Levels and the Effect of Dephosphorylation
on the Microtubule Promoting Activity of AD
and Control (Ct) Brain Cytosols

| | ^{a}N | | ^{c}Assemb. | |
|---|---|---|---|---|
| | tau (cpm) | ^{b}P-tau (%) | (cpm) | ^{d}Inc. Assemb. (%) |
| AD(6) | 1910 ± 458^{f} | 77 ± 53^{g} | 3800 ± 1300^{h} | 70 ± 29ⁱ |
| Ct(6) | 2923 ± 649^{f} | 1.8 ± 75^{g} | 6330 ± 1134^{h} | 2.2 ± 9.7ⁱ |

^{a}The concentration of normal tau (N tau) and abnormally phosphorylated tau (P-tau) were determined by radioimmunoassay (Khatoon et al., 1992, J. Neurochem. 59:750-753). The values of N tau are expressed as the amount of radioactivity bound per 1 g of cytosol protein; P-tau values are expressed as % of N tau. ^{c}Fresh rat brain tubulin was incubated for 20 min at 37° C. with cytosolic extracts from control and AD brains in the polymerization buffer. The samples were then centrifuged at 100,000×g for 30 min at 37° C., and the pellets were dissolved in 50 mM MES buffer, kept in ice for 30 min, and centrifuged at 100,000×g at 4° C., and the amounts of tubulin present in these supernatants were assayed by radioimmuno-slot-blot using DM1-A tubulin antibody The amount of assembly is expressed as cpm. ^{d}The increase in the amount of tubulin assembly with the alkaline phosphatase-treated cytosols was calculated by taking the value obtained without dephosphorylation as 100%. p<0.0l; gp<0.004; lp<0.006; p<0.004; using the nonparametric Mann-Whitney U test.

Because it is well known that tubulin in frozen tissue loses its ability to polymerize, we added fresh rat brain tubulin to a cytosolic fraction of either AD or control brain and assayed polymerization of tubulin; the concentration of tau in both cytosols was adjusted to the same level by dilution with the buffer. A high background resulting from the use of cytosol did not allow a reliable measure of turbidimetric changes, and therefore, the polymerization of tubulin was measured by immuno-assaying the amount of the cold-disassembled protein following the assembly at 37° C. for 20 minutes. The cytosolic fraction of AD brain was effective in promoting microtubule assembly, although this activity was approximately 60% less than that of the control cytosolic fraction, as judged by the amount of tubulin in the cold-disassembled fraction obtained after the incubation (Table 5).

Dephosphorylation with alkaline phosphatase treatment dramatically increased the microtubule assembly-promoting activity of AD cytosols, but this increase was negligible in control cases (Table 5). The microtubules obtained with both preparations were of similar length and appearance (figure not shown).

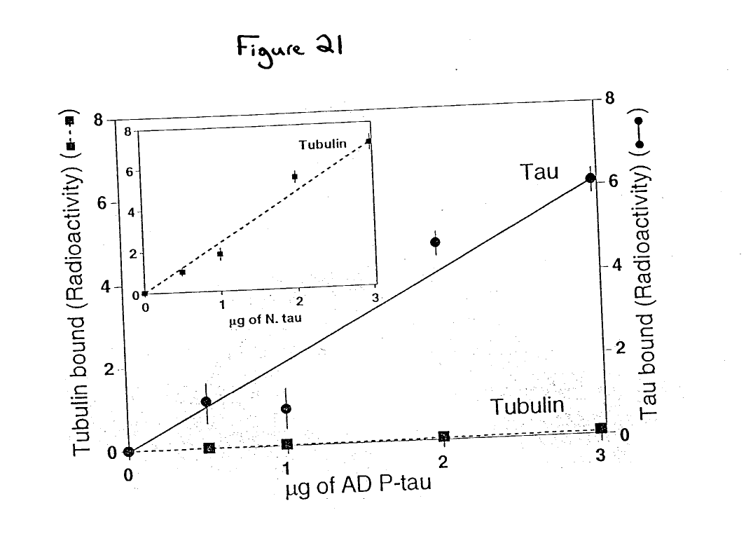

AD P-tau Inhibits Microtubule Assembly and Binds to Normal Tau. Because even after adjusting the normal tau levels to control values the AD brain cytosol was significantly less (i.e., 40%) active than the corresponding control fraction in promoting assembly of microtubules (Table 3), we investigated further whether and how AD P-tau inhibited the activity of normal tau. Different concentrations of AD P-tau were added to normal tau before it was mixed with tubulin, and the assembly was determined as described above. AD P-tau inhibited microtubule assembly, and this inhibition was almost total when the concentration of AD P-tau was two times 1:; that of normal tau (FIG. 20).

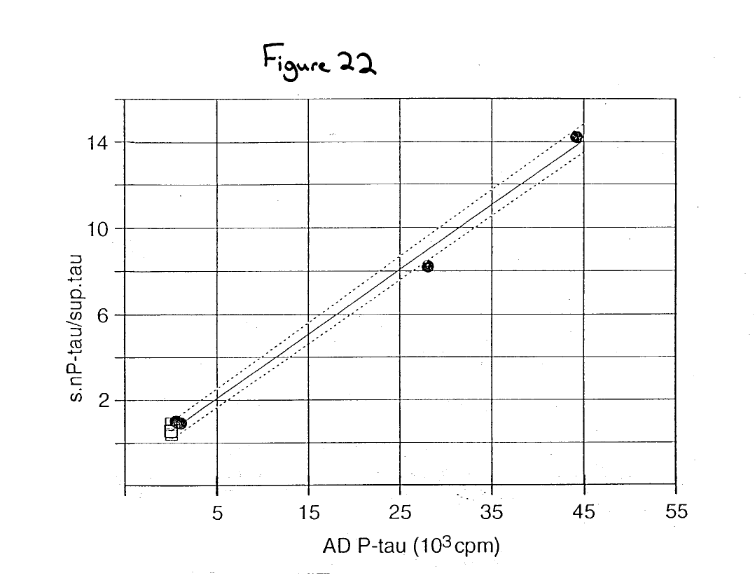

To study if the inhibition observed with AD P-tau was caused by its interaction with tubulin or normal tau, protein-binding studies were carried out by an overlay dot assay. AD P-tau was dotted on a nitrocellulose paper and overlaid either with tubulin or normal tau, followed by an incubation with anti-tubulin antibody or Tau-1 antibody. In the case of the strip overlaid with tubulin, there was no detectable binding, whereas there was a considerable binding of normal human tau to AD P-tau (FIG. 21) In these assays, tubulin was bound to normal tau when it was dotted (FIG. 21, inset), and no binding was observed when bovine serum albumin was used as a negative control (figure not shown). These studies suggest that AD P-tau inhibits the microtubule assembly probably through its interaction with normal tau, and not with tubulin.

Levels of Sedimentable Tau Correlate with Levels of AD P-tau.

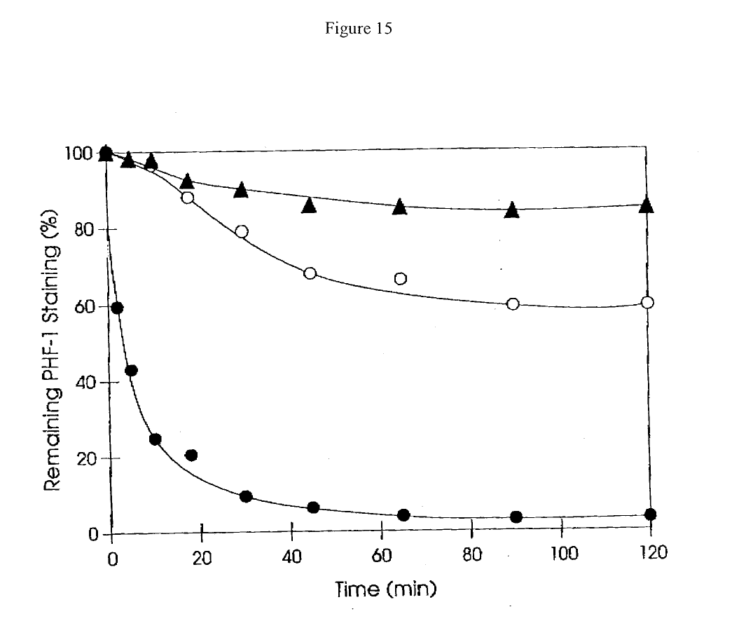

Previously, we have shown that some of the tau that is non-phosphorylated at Tau-1 epitope sediments at 200,000×g and the levels of this sedimentable tau are markedly higher in AD than in control brains (Köpke et al., 1993, J. Biol. Chem. 268:24374-24384). In light of our findings in the present study on the binding of AD P-tau to normal tau, we investigated whether the 27,000×g to 200,000×g fraction where the soluble AD P-tau sediments also contains proportionally higher levels of the non-hyperphosphorylated tau. We determined the levels of the non-hyperphosphorylated tau and AD P-tau in the 27,000×g-200,000×g fraction and tau in 200,000×g supernatants from four AD cases We also carried out the above studies on four control cases. The levels of the non-hyperphosphorylated tau showed a direct correlation with the levels of AD P-tau in the 27,000 to 200,000×g fraction, whereas the levels of tau in the 200,000×g supernatant had an inverse correlation Therefore, the ratio of the sedimentable non-hyperphosphorylated tau to tau in the supernatant directly and strongly correlated with the amount of AD P-tau in the 27,000 to 200,000×g fraction (FIG. 22). The control brains did not contain any detectable levels of the abnormally phosphorylated tau and had only background levels of tau in the 27,000×g to 200,000×g fraction. These findings are consistent with the studies in the previous section showing that AD P-tau binds to normal tau.

### 9.3. Discussion

In the present study, we have investigated a cause and a mechanism of this breakdown of the microtubule system in-neurons with NFT of PHF in AD brains. We have found (i) that the abnormally phosphorylated tau is functionally inactive in binding to tubulin and stimulating the assembly of microtubules, (ii) that the microtubule assembly-promoting activity of the abnormal tau is restored by dephosphorylation, (iii) that levels of normal/functional tau in brain cytosol of AD cases are approximately 35% lower than those in non-AD control cases, (iv) that the abnormally phosphorylated tau inhibits tau-promoted assembly of tubulin into microtubules, and (v) that the abnormal tau binds to normal tau and not to tubulin, suggesting that it inhibits the assembly by interacting with normal tau.

In AD brains, the levels of tau are several-fold higher than in age-matched control brains, and this increase is in the form of the abnormally phosphorylated protein (Khatoon et al., 1992, J. Neurochem. 59:750-753). However, in the present study, when tau was isolated from AD brains with 2.5% HClO₄ extraction, only non-abnormally phosphorylated tau was obtained. AD P-tau is probably denatured by 2.5% HClO₄ treatment and is not extracted. This finding is in agreement with our previous observations (Köpke et al., 1993, J. Biol. Chem. 268:24374-24384). Although the yield of the acid-soluble tau from AD brains was approximately 70% of that of the control cases, the microtubule-promoting activity of this tau was not significantly different from that of control tau, both in the total amount of microtubules formed and the rates of assembly and disassembly.

On the other hand, AD P-tau isolated from AD cases showed minimal, if any, microtubule-promoting activity. When this tau was dephosphorylated, the activity increased to approximately the same level as occurs with the acid-soluble tau. These results suggest that the abnormal phosphorylation of tau diminishes its microtubule-promoting activity, which can be recovered after dephosphorylation. Dephosphorylation of AD brain cytosol with alkaline phosphatase led to an increase in the microtubule-promoting activity, suggesting that AD P-tau in the extract also could be reactivated by dephosphorylation. However, in a brain cytosolic extract tau is not the only protein that can promote microtubule assembly; microtubule associated protein 2(MAP 2) might also be present, and its activity is also modulated by its degree of phosphorylation. Thus, the increase in microtubule assembly obtained with the dephosphorylated cytosol cannot rule out the involvement of proteins in addition to tau. Recovery of tau activity by dephosphorylation was also obtained with PBF-tau by Iqbal et al. (1991, J. Neuropathol. Exp. Neurol. 50:316 (Abstract)) and Bramblett et al. (1993, Neuron 10:1089-1099); the latter study, however, employed the binding of tau to taxol-stabilized microtubules, and not the microtubule assembly promoting activity.

Because AD P-tau has minimal microtubule-promoting activity, this protein did not contribute to the assembly-promoting activity found when AD cytosol extracts were used, although AD P-tau is present there in considerable amounts. Furthermore, we suspected that the altered protein could be inhibiting the assembly because the levels of microtubules formed with AD extracts were lower than those formed with control extracts. The putative inhibitory effect of AD P-tau was confirmed in a system of purified tubulin and normal tau in which AD P-tau inhibited the tau-promoted assembly of tubulin. This inhibitory effect of AD P-tau might be the reason for the low level of polymerization found with AD cytosolic extract.

We studied the mechanism by which AD P-tau might be inhibiting the microtubule assembly, testing the interactions between AD P-tau, normal tau, and tubulin. We found that AD P-tau was able to bind normal tau and not tubulin. These results indicate that the inhibition of microtubule assembly might be caused by an interaction of AD P-tau with normal tau in the purified system. It is also possible that the inhibition seen in the assembly with the AD cytosolic extracts is the result of an interaction of AD P-tau with normal tau. This possibility is supported by the findings of Iqbal et al. (1986,The Lancet 421:426), who were able to see polymerization of tubulin in AD extracts when they replaced tau with DEAE-dextran, showing that in AD brains tubulin is not compromised and is able to polymerize.

In conclusion, the present study suggests that the abnormal phosphorylation of tau probably causes microtubule disruption by decreasing the levels of functional tau in two ways: (i) directly, by diminishing its microtubule-promoting activity and (ii) indirectly, by binding to normal tau and making it unavailable for promoting microtubule assembly. Dephosphorylation restores this tau functional deficit. It appears that avoiding the hyperphosphorylation of tau can result in the prevention of microtubule disruption in neurons with neurofibrillary degeneration in AD.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

Various publications are cited herein, the disclosures of which are incorporated by reference in their entireties.

What is claimed is:

1. A method of treating a subject having a disease or disorder associated with the presence of neurofibrillary tangles comprising administering to the subject a therapeutically effective amount of a composition comprising a molecule, which molecule increases the activity of at least one protein phosphatase towards abnormal hyperphosphorylated tau, which protein phosphatase is selected from the group consisting of PP-2A, PP-2B, PP-1, and related protein phosphatases.

2. The method according to claim 1 in which the disease or disorder is Alzheimer disease.

3. The method according to claim 1 in which the molecule is selected from the group consisting of manganese, calcium, and polylysine.

4. The method according to claim 2 in which the molecule is selected from the group consisting of manganese, calcium, and polylysine.

5. The method according to claim 3 in which the molecule is manganese, in salt, conjugate, or ionic form, with the proviso that the molecule is not manganese pyruvate or a manganese chelate of an alkylamino-ester of phosphoric acid.

6. The method according to claim 4 in which the molecule is manganese, in salt, conjugate, or ionic form, with the proviso that the molecule is not manganese pyruvate or a manganese chelate of an alkylamino-ester of phosphoric acid.

7. The method according to claim 4 in which the molecule is selected from the group consisting of manganese chloride, manganese sulfate, manganese acetate, manganese gluconate, manganese lactate and manganese citrate.

8. The method according to claim 7 in which said administering is oral.

9. The method according to claim 1 in which the protein phosphatase is PP-2B.

10. The method according to claim 1 in which the protein phosphatase is PP-2A.

11. The method according to claim 1 in which the protein phosphatase is PP-1.

12. The method according to claim 1 in which the protein phosphatase is PP-2B, PP-2A, and PP-1.

13. The method according to claim 1 or 2 in which the subject is human.

14. The method according to claim 6 or 7 in which the subject is human.

15. A method of treating a subject having a disease or disorder associated with the presence of neurofibrillary tangles comprising administering to the subject a therapeutically effective amount of a composition comprising at least one protein phosphatase which dephosphorylates abnormal hyperphosphorylated tau, which protein phosphatase is selected from the group consisting of PP-2A, PP-2B, PP-1, and related protein phosphatases.

16. The method according to claim 15 in which the disease or disorder is Alzheimer disease.

17. The method according to claim 15 in which the phosphatase is PP-2B.

18. The method according to claim 16 in which the phosphatase is PP-2B.

19. The method according to claim 15 in which the phosphatase is PP-2A.

20. The method according to claim 16 in which the phosphatase is PP-2A.

21. The method according to claim 15 in which the phosphatase is PP-1.

22. The method according to claim 16 in which the phosphatase is PP-1.

23. The method according to claim 16 in which the composition comprises PP-2B, PP-2A, and PP-1.

24. The method according to claim 15 or 16 in which the subject is human.

25. A method of treating a subject having a disease or disorder associated with the presence of neurofibrillary tangles comprising administering to the subject a therapeutically effective amount of a composition comprising a nucleic acid encoding a protein phosphatase which dephosphorylates abnormal hyperphosphorylated tau, which protein phosphatase is selected from the group consisting of PP-2A, PP-2B, PP-1, and related protein phosphatases.

26. The method according to claim 25 in which the disease or disorder is Alzheimer disease.

27. The method according to claim 25 in which the phosphatase is PP-2B.

28. The method according to claim 26 in which the phosphatase is PP-2B.

29. The method according to claim 25 in which the phosphatase is PP-2A.

30. The method according to claim 26 in which the phosphatase is PP-2A.

31. The method according to claim 25 in which the phosphatase is PP-1.

32. The method according to claim 26 in which the phosphatase is PP-1.

33. The method according to claim 25 or 26 in which the subject is human.

34. A pharmaceutical composition comprising a therapeutically effective amount of a phosphatase which dephosphorylates abnormal hyperphosphorylated tau, and a pharmaceutically acceptable carrier; in which the protein phosphatase is selected from the group consisting of PP-2A, PP-2B, PP-1, and related protein phosphateses.

35. The composition of claim 34 in which the phosphatase is PP-2B.

36. The composition of claim 34 in which the phosphatase is PP-2A.

37. The composition of claim 34 in which the phosphatase is PP-1.

38. The composition of claim 34 in which the composition comprises PP-2B, PP-2A, and PP-1.

39. A pharmaceutical composition comprising a therapeutically effective amount of a nucleic acid encoding a phosphatase which dephosphorylates abnormal hyperphosphorylated tau, and a pharmaceutically acceptable carrier; in which the protein phosphatase is selected from the group consisting of PP-2A, PP-2B, PP-1, and related protein phosphatases.

40. The composition of claim 39 in which the phosphatase is PP-2B.

41. The composition of claim 39 in which the phosphatase is PP-2A.

42. The composition of claim 39 in which the phosphatase is PP-1.

43. A pharmaceutical composition comprising a therapeutically effective amount of a manganese ion, manganese salt, or manganese conjugate, in a slow-release formulation.

44. A method of diagnosing the presence in a subject of a disease or disorder associated with the presence of neurofibrillary tangles comprising:
(a) contacting a sample derived from the subject with an antibody to a phosphorylated epitope of abnormal hyperphosphorylated tau under conditions such that immunospecific binding can occur; and
(b) detecting or measuring the amount of any immunospecific binding which occurs of a component in the sample to the antibody,
in which increased levels of immunospecific binding relative to the level of immunospecific binding which occurs in a subject not having the disease or disorder, indicates the presence of the disease or disorder in the subject.
